(19) United States
(12) Patent Application Publication
(10) Pub. No.: US 2024/0201575 A1
(43) Pub. Date: Jun. 20, 2024

(54) PHOTOSENSITIVE SURFACE TREATMENT AGENT, LAMINATE, TRANSISTOR, METHOD FOR FORMING PATTERN AND METHOD FOR MANUFACTURING TRANSISTOR

(71) Applicant: NIKON CORPORATION, Tokyo (JP)

(72) Inventors: Yusuke KAWAKAMI, Yokohama-shi (JP); Kazuo YAMAGUCHI, Mitaka-shi (JP); Michiko ITOU, Yokohama-shi (JP)

(73) Assignee: NIKON CORPORATION, Tokyo (JP)

(21) Appl. No.: 18/443,248

(22) Filed: Feb. 15, 2024

Related U.S. Application Data
(63) Continuation of application No. PCT/JP2022/030265, filed on Aug. 8, 2022.

(30) Foreign Application Priority Data
Aug. 17, 2021 (JP) 2021-132671

(51) Int. Cl. G03C 1/72; C07C 323/16; C08F 20/38; G03F 7/004; G03F 7/38; H01L 21/288; H01L 29/66; H01L 29/786; H05K 1/03; H10K 71/60; H10K 85/60

(52) U.S. Cl. CPC G03C 1/72; C07C 323/16; C08F 20/38; G03F 7/004; G03F 7/38; H01L 21/288; H01L 29/66477; H01L 29/786; H05K 1/03; H10K 71/60; H10K 85/60

(57) ABSTRACT

A photosensitive surface treatment agent containing a compound represented by the following formula (1) or a polymer compound derived from the following formula (1).

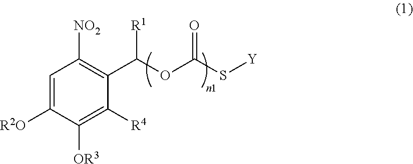

### TECHNICAL FIELD

The present invention relates to a photosensitive surface treatment agent, a laminate, a transistor, a method for forming a pattern and a method for manufacturing a transistor.

Priority is claimed on Japanese Patent Application No. 2021-132671, filed Aug. 17, 2021, the content of which is incorporated herein by reference.

### BACKGROUND ART

In recent years, in the manufacturing of fine devices and the like such as semiconductor elements, integrated circuits and devices for organic EL displays, a method in which a pattern with different surface characteristics is formed on a substrate and a fine device is produced using the difference of the surface characteristics has been proposed.

As a method for forming a pattern using a difference of the surface characteristics on a substrate, for example, there is a method in which a region where a chemically active substituent has been generated is formed in a part of a substrate. This method makes it possible to attach a metallic material, an organic material or an inorganic material to a part of the substrate.

As a technique for forming a metal film by attaching a metallic material onto a substrate, there is an electroless plating treatment. For example, Patent Document 1 discloses a technique for forming a fine wiring by the electroless plating treatment. Specifically, Patent Document 1 discloses that etching or photopatterning by lift-off is performed on one surface in a plated state using a catalyst activation layer and a photoresist.

### CITATION LIST

### Patent Document

### [Patent Document 1]

Japanese Unexamined Patent Application, First Publication No. 2006-2201

### SUMMARY OF INVENTION

### Technical Problem

A first aspect of the present invention is a photosensitive surface treatment agent containing a compound represented by the following formula (1) or a polymer compound derived from the following formula (1).

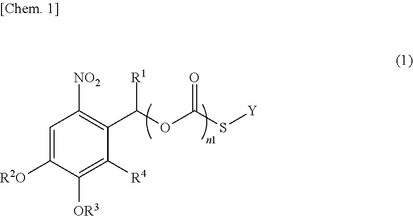

[In the formula (1), R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen atom or a nitro group, n1 is 0 or 1, and Y is a polymerizable group-containing group or a linear or branched alkyl group having 1 to 20 carbon atoms.]

Another aspect of the present invention is a transistor containing a compound represented by the following formula (1) or a polymer compound derived from the following formula (1).

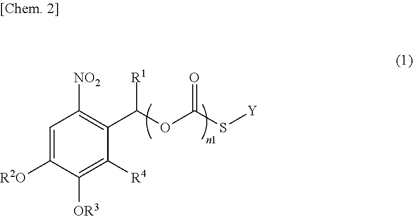

[In the formula (1), R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen atom or a nitro group, n1 is 0 or 1, and Y is a polymerizable group-containing group or a linear or branched alkyl group having 1 to 20 carbon atoms.]

### BRIEF DESCRIPTION OF DRAWINGS

(a) to (f) of FIG. 1 is pattern diagrams showing a method for forming a pattern of the present embodiment.

(a) to (d) of FIG. 2 is pattern diagrams showing a method for manufacturing a transistor of the present embodiment.

FIG. 3 is overall photographs and optical microscope photographs of a substrate on which a plating treatment has been performed using a photosensitive surface treatment agent of the present embodiment.

### DESCRIPTION OF EMBODIMENTS

### Photosensitive Surface Treatment Agent

A photosensitive surface treatment agent of the present embodiment contains a compound represented by the following formula (1) or a polymer compound derived from the following formula (1).

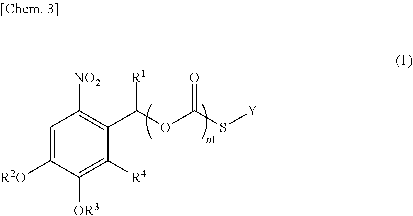

[In the formula (1), R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen atom or a nitro group, n1 is 0 or 1, and Y is a polymerizable group-containing group or a linear or branched alkyl group having 1 to 20 carbon atoms.]

### Compound Represented by Formula (1)

When the photosensitive surface treatment agent containing the compound represented by the formula (1) is applied onto a substrate and irradiated with light, a nitrobenzyl group is desorbed, Y is attached to the substrate, and a thiol group (—SH) is generated at the same time. To a portion where the thiol group has been generated, it is possible to attach a metallic material, an organic material or an inorganic material.

According to the photosensitive surface treatment agent of the present embodiment, a metal pattern having a line width of 5 μm or less can be formed on the surface of the substrate without using a photoresist step, a development step and an etching step by disposing a metallic material on the thiol generation part formed on the substrate surface.

### (R¹)

In the formula (1), R¹ is a hydrogen or an alkyl group having 1 to 5 carbon atoms. Examples of the alkyl group having 1 to 5 carbon atoms include a methyl group, an ethyl group, a propyl group, an isopropyl group, a n-butyl group and a tert-butyl group, and, among these, a methyl group or an ethyl group is preferable and a methyl group is more preferable.

(R² and R³)

R² and R³ are each independently an alkyl group having 1 to 3 carbon atoms or a fluoroalkyl group having 1 to 13 carbon atoms. Examples of the alkyl group having 1 to 3 carbon atoms include a methyl group, an ethyl group and a propyl group, a methyl group or an ethyl group is preferable, and a methyl group is more preferable.

Examples of the fluoroalkyl group having 1 to 13 carbon atoms include groups obtained by substituting a part or all of the hydrogen atoms of a methyl group, an ethyl group, a propyl group, a heptyl group, a nonyl group, an undecyl group or a tridecyl group, groups including a perfluoroalkyl group having 4 to 10 carbon atoms are preferable, and nonyl groups including a perfluoroalkyl group having 6 carbon atoms are more preferable.

### (R⁴)

In the formula (1), R⁴ is a hydrogen atom or a nitro group.

(n1)

In the formula (1), n1 is 0 or 1.

### (Y)

In the formula (1), Y is a polymerizable group-containing group or a linear or branched alkyl group having 1 to 20 carbon atoms.

“Polymerizable group” is a group enabling a compound having a polymerizable group to polymerize by radical polymerization or the like and refers to, for example, a group including a multiple bond between carbon atoms such as an ethylenic double bond.

Examples of the polymerizable group include a vinyl group, an allyl group, an acryloyl group, a methacryloyl group and the like.

“Polymerizable group-containing group” is a group including a polymerizable group. The polymerizable group-containing group may be a group composed of a polymerizable group alone or may be a group composed of a polymerizable group and a group other than polymerizable groups.

As Y, the following formula (Y1) is an exemplary example.

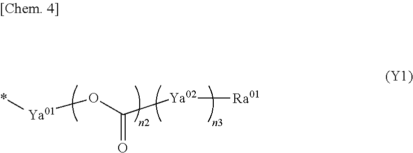

[In the formula (Y1), Ya⁰¹ is an alkylene group having 1 to 10 carbon atoms, Ya⁰² is a group obtained by removing two hydrogen atoms from an aromatic ring having 6 to 15 carbon atoms, Ra⁰¹ is a polymerizable group or a linear or branched alkyl group having 1 to 20 carbon atoms and n2 and n3 are each independently 0 or 1. * means a bonding site to a sulfur element.]

### (Ya⁰¹)

In the formula (Y1), Ya⁰¹ is an alkylene group having 1 to 10 carbon atoms.

Ya⁰¹ is preferably a linear or branched alkylene group. Examples of the linear alkylene group having 1 to 10 carbon atoms include a methylene group [—CH₂—], an ethylene group [—(CH₂)₂—], a trimethylene group [—(CH₂)₃—], a tetramethylene group [—(CH₂)₄—], a pentamethylene group [—(CH₂)₅—] and the like.

Examples of the branched alkylene group having 1 to 10 carbon atoms include alkylalkylene groups such as alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)— and —C(CH₂CH₃)₂—; alkylethylene groups such as —CH(CH₃)CH₂—, —CH(CH₃)CH(CH₃)—, —C(CH₃)₂CH₂—, —CH(CH₂CH₃)CH₂— and —C(CH₂CH₃)₂—CH₂—; alkyltrimethylene groups such as —CH(CH₃)CH₂CH₂— and —CH₂CH(CH₃)CH₂—; alkyltetramethylene groups such as —CH(CH₃)CH₂CH₂CH₂— and —CH₂CH(CH₃)CH₂CH₂—; and the like. An alkyl group in the alkylalkylene groups is preferably a linear alkyl group having 1 to 5 carbon atoms.

### (Ya⁰²)

In the formula (Y1), Ya⁰² is a group obtained by removing two hydrogen atoms from an aromatic ring having 6 to 15 carbon atoms.

Specific examples of Ya⁰² include groups obtained by removing two hydrogen atoms from a benzene ring, a fluorene ring, a naphthalene ring or an anthracene ring.

### (Ra⁰¹)

In the formula (Y1), Ra⁰¹ is a polymerizable group or a linear or branched alkyl group having 1 to 20 carbon atoms.

The linear alkyl group is preferably a linear alkyl group having 3 to 18 carbon atoms, and the number of carbon atoms is more preferably 5 to 15 and still more preferably 6 to 12.

Examples of the branched alkyl group include branched alkyl groups having 3 to 20 carbon atoms. The number of carbon atoms in the branched alkyl group is preferably 3 to 18 and more preferably 3 to 15.

In the formula (Y1), n2 and n3 are each independently 0 or 1. * means a bonding site to a sulfur element.

As Y, the following formula (Y2) is an exemplary example.

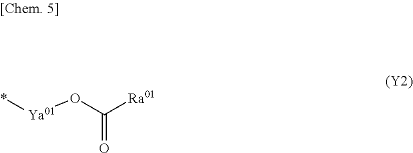

[In the formula (Y2), Ya⁰¹ means an alkylene group having 1 to 10 carbon atoms, Ra⁰¹ means a polymerizable group or a linear or branched alkyl group having 1 to 20 carbon atoms, and * means a bonding site to a sulfur element.]

Description relating to Y⁰¹ and Ra⁰¹ in the formula (Y2) is the same as described above.

As Y, the following formula (Y3) is an exemplary example.

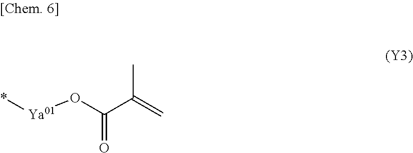

[In the formula (Y3), Ya⁰¹ means an alkylene group having 1 to 10 carbon atoms and * means a bonding site to a sulfur element.]

Description relating to Y⁰¹ in the formula (Y3) is the same as described above.

As Y, the following formula (Y3-2) is an exemplary example.

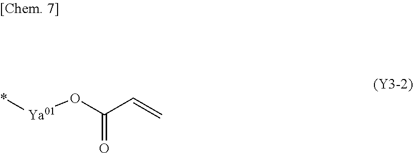

[In the formula (Y3-2), Ya⁰¹ means an alkylene group having 1 to 10 carbon atoms and * means a bonding site to a sulfur element.]

Description relating to Ya⁰¹ in the formula (Y3-2) is the same as described above.

As Y, the following formula (Y4) is an exemplary example.

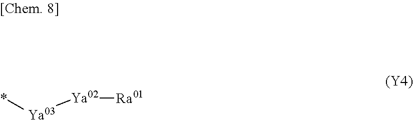

[In the formula (Y4), Ya⁰³ means an alkylene group having 1 to 10 carbon atoms that may have an ether bond, Ya⁰² means a group obtained by removing two hydrogen atoms from an aromatic ring having 6 to 15 carbon atoms, Ra⁰¹ means a polymerizable group or a linear or branched alkyl group having 1 to 20 carbon atoms, and * means a bonding site to a sulfur element.]

Description relating to Y⁰² and Ra⁰¹ in the formula (Y4) is the same as described above.

In the formula (Y4), Ya⁰³ is an alkylene group having 1 to 10 carbon atoms that may have an ether bond (—O—). Ya⁰³ is preferably an alkylene group having 1 to 10 carbon atoms or a group composed of a combination of an ether bond (—O—) and an alkylene group having 1 to 10 carbon atoms.

As Y, the following formula (Y5) is an exemplary example.

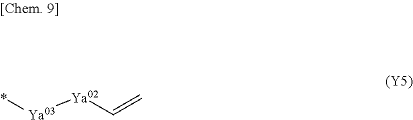

[In the formula (Y5), Ya⁰³ means an alkylene group having 1 to 10 carbon atoms that may have an ether bond, Ya⁰² means a group obtained by removing two hydrogen atoms from an aromatic ring having 6 to 15 carbon atoms, and * means a bonding site to a sulfur element.]

Description relating to Y⁰² and Ya⁰³ in the formula (Y5) is the same as described above.

As Y, the following formula (Y5-2) is an exemplary example.

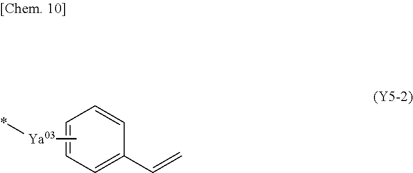

[In the formula (Y5-2), Ya⁰³ means alkylene having 1 to 10 carbon atoms that may have an ether bond, and * means a bonding site to a sulfur element.]

Description relating to Ya⁰³ in the formula (Y5-2) is the same as described above.

Specific examples of the compound represented by the formula (1) will be shown below.

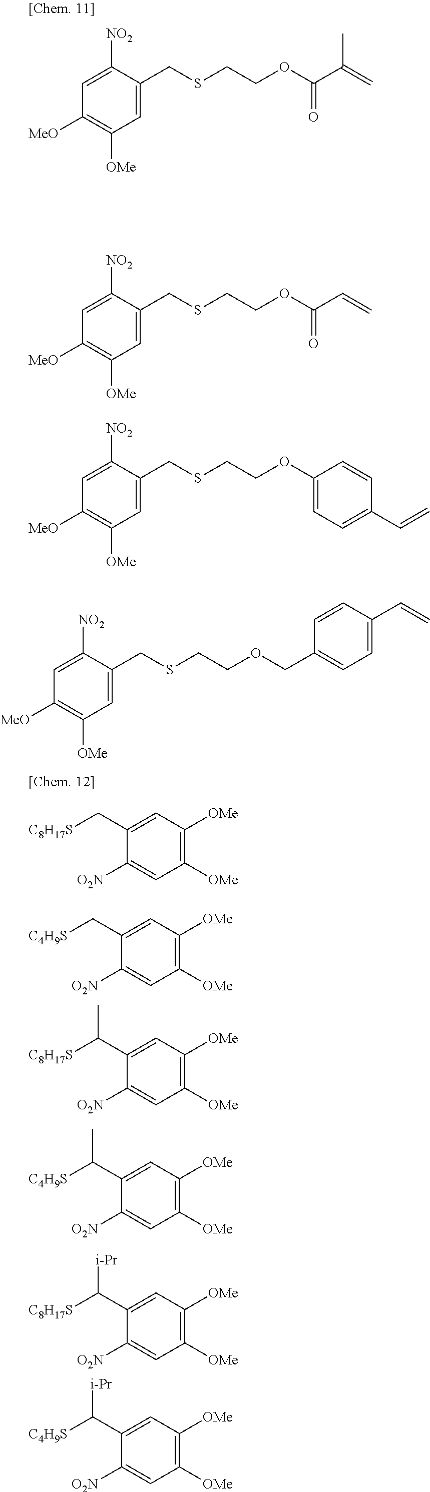

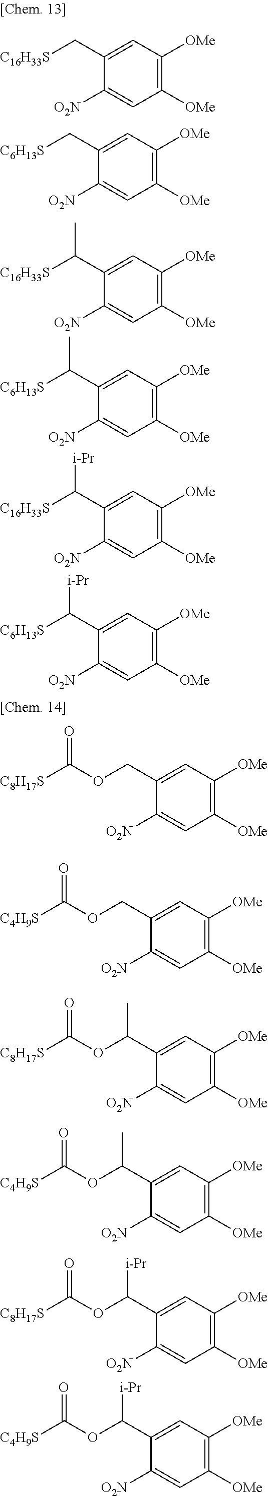

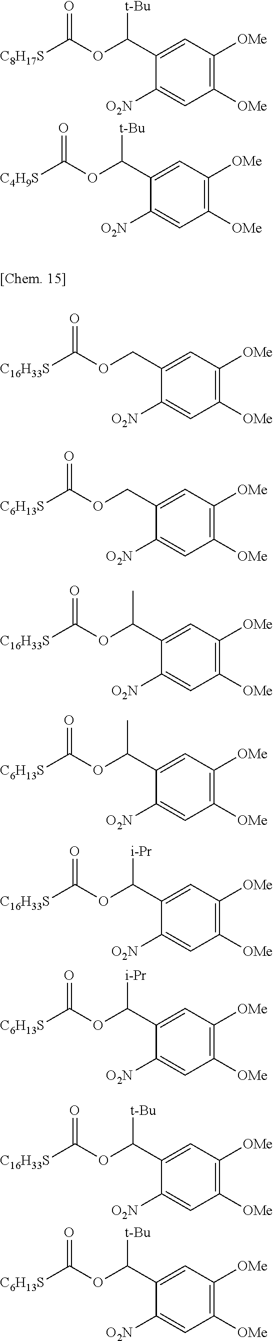

### Polymer Compound Derived from Compound Represented by Formula (1)

The polymer compound derived from the compound represented by the formula (1) is a polymer compound in which the polymerizable group in the compound represented by the formula (1) has been converted into a main chain.

“The polymerizable group has been converted into a main chain” refers to the fact that the multiple bond in the polymerizable group is cleft to form a main chain. For example, in the case of a monomer having an ethylenic double bond, it means that the ethylenic double bond is cleft and a single bond between carbon atoms form a main chain.

The polymer compound derived from the compound represented by the formula (1) is specifically represented by the following formula (1)-1.

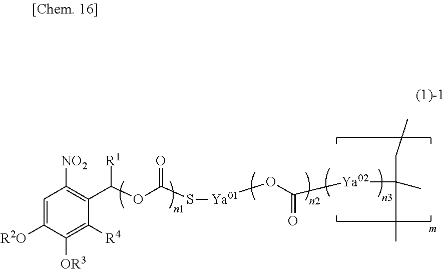

[In the formula (1)-1, R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen or a nitro group, Ya⁰¹ is an alkylene group having 1 to 10 carbon atoms, Ya⁰² is a group obtained by removing two hydrogen atoms from an aromatic ring having 6 to 15 carbon atoms, n1, n2 and n3 are each independently 0 or 1, and m is a natural number.]

R¹, R², R³, R⁴, Ya⁰¹, Ya⁰², n1, n2 and n3 in the formula (1)-1 are the same as described above, and m is a natural number.

In the polymer compound derived from the compound represented by the formula (1), it is preferable that a substituent represented by the following formula (1x) bonds to at least one end of the main chain. In the following formula (1x), * means a bonding site to an end of the main chain of the polymer compound derived from the compound represented by the formula (1).

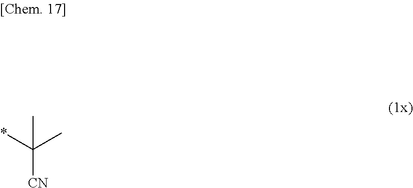 [SMILES: *C(C)(C)C#N]

An exemplary example of polymer compounds in which the substituent represented by the formula (1x) bonds to one end of the main chain of the polymer compound represented by the formula (1)-1 is shown below.

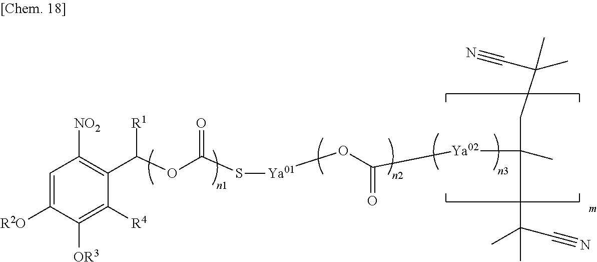

Other specific examples of the polymer compound represented by the formula (1)-1 will be shown below.

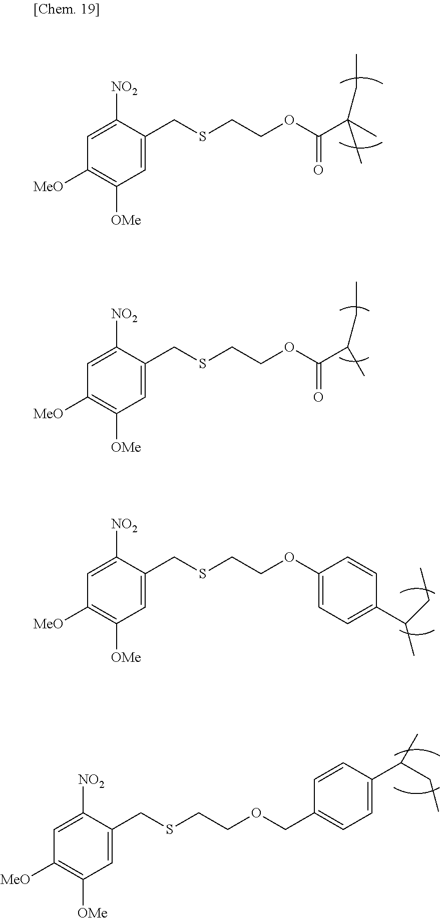

The number-average molecular weight of the polymer compound derived from the compound represented by the formula (1) is preferably 300 to 100,000.

In one aspect of the present invention, the photosensitive surface treatment agent is composed of the compound represented by the formula (1).

In one aspect of the present invention, the photosensitive surface treatment agent is composed of the polymer compound represented by the formula (1)-1.

In one aspect of the present invention, the photosensitive surface treatment agent is composed of the polymer compound represented by the formula (1) and the compound represented by the formula (1)-1.

In one aspect of the present invention, the photosensitive surface treatment agent may contain a solvent. Examples of the solvent that the photosensitive surface treatment agent may contain include cyclopentanone, cycloheptanone, N-methyl-2-pyrrolidone (NMP), cyclohexanone and the like, and, among these, cyclopentanone is preferable.

### Method for Manufacturing Compound

The compound represented by the formula (1) can be manufactured by the following method.

In the description of the following manufacturing method, description relating to each reference symbol in formulae is the same as described above.

### [Manufacturing Method 1]

The compound represented by the formula (1) is manufactured by reacting an alcohol body represented by the following formula (M1) and methacryloyl chloride or acryloyl chloride.

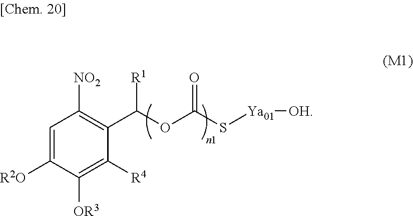

Specific example 1 of the manufacturing method 1 will be shown below.

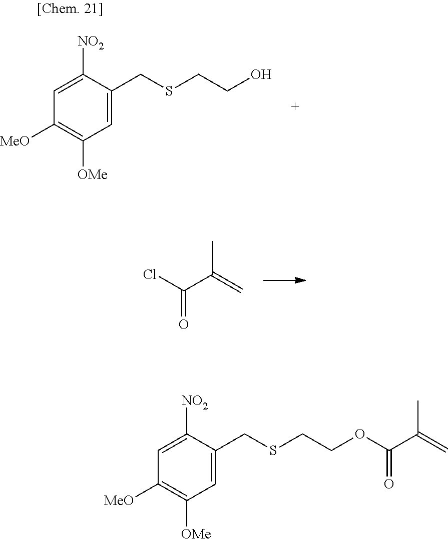

Specific example 2 of the manufacturing method 1 will be shown below.

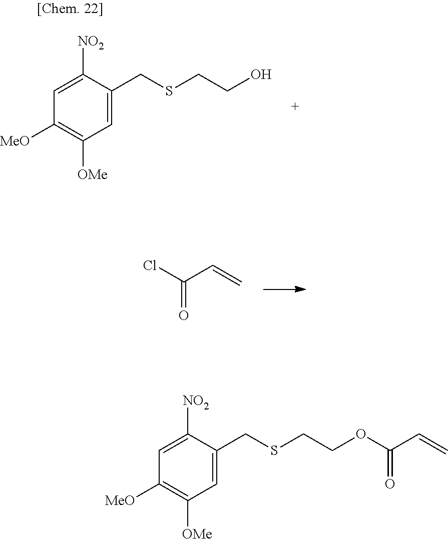

Specific example 3 of the manufacturing method 1 will be shown below.

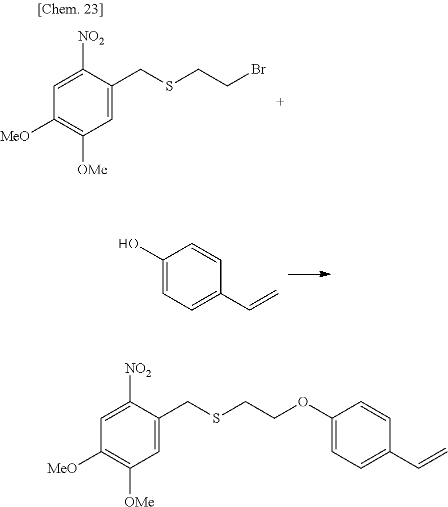

Specific example 4 of the manufacturing method 1 will be shown below.

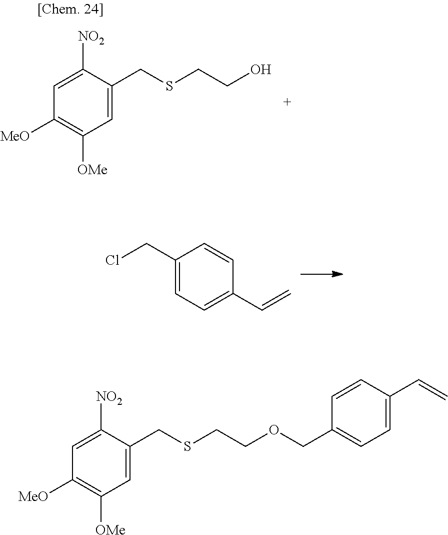

### [Manufacturing Method 2]

An intermediate (M2)-1 is obtained by reacting an alcohol body represented by the following formula (M2) and succinimidyl carbonate.

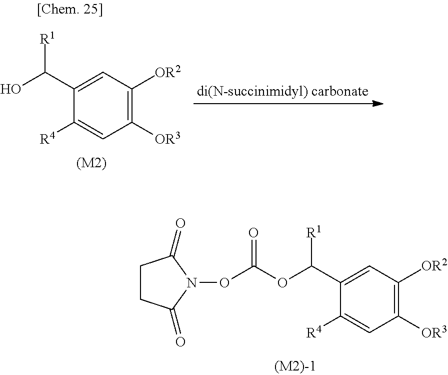

A compound represented by a formula (1)-A is obtained by reacting 1-octanethiol with the intermediate (M2)-1. At this time, for example, 1-butanethiol (having 4 carbon atoms), 1-hexanethiol (having 6 carbon atoms), 1-hexadecanethiol (having 16 carbon atoms) or 1-docosanethiol (having 22 carbon atoms) may be reacted instead of 1-octanethiol.

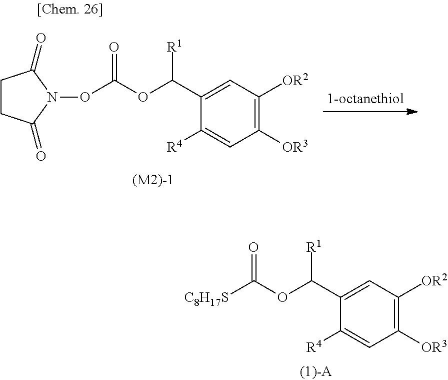

### [Manufacturing Method 3]

An intermediate (M3)-1 is obtained by reacting an alcohol body represented by the following formula (M3), triphenylphosphine and carbon tetrabromide. At this time, the intermediate (M3)-1 may be obtained by reacting the alcohol body represented by the following formula (M3) and phosphorus tribromide.

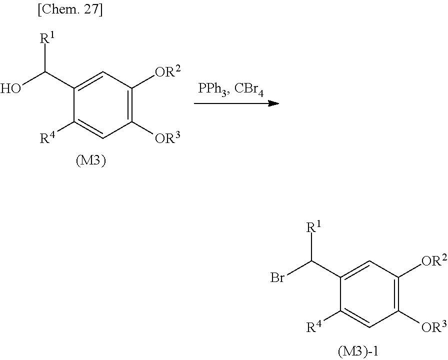

A compound represented by a formula (1)-B is obtained by reacting 1-octanethiol with the intermediate (M3)-1. At this time, for example, 1-butanethiol (having 4 carbon atoms), 1-hexanethiol (having 6 carbon atoms), 1-hexadecanethiol (having 16 carbon atoms) or 1-docosanethiol (having 22 carbon atoms) may be reacted instead of 1-octanethiol.

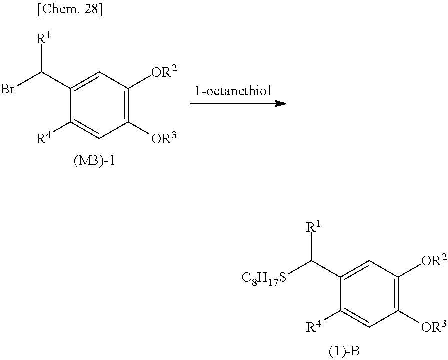

### Method for Forming Pattern

A method for forming a pattern of the present embodiment includes a step of applying the photosensitive surface treatment agent of the present embodiment onto a substrate to form a photosensitive resin film, a step of irradiating the photosensitive resin film with light of a predetermined pattern to form a thiol generation region in an exposure region and a step of disposing a catalyst for electroless plating in the thiol generation region and performing electroless plating.

Hereinafter, each step will be described with reference to drawings.

As shown in (a) of FIG. 1, a photosensitive surface treatment agent 10a of the present embodiment is applied onto a substrate 11.

As an application method, for example, application methods such as a spin coating method, a dip coating method, a die coating method, a spray coating method, a roll coating method and brush coating can be used. In addition, the photosensitive surface treatment agent may be applied by a printing method such as flexographic printing or screen printing.

In the present step, a step of drying a solvent with, for example, heat, decompression or the like may be added as shown in (a) of FIG. 1.

Therefore, a photosensitive surface treatment agent layer 10 is formed on the substrate 11 as shown in (b) of FIG. 1.

Next, a photomask 13 having exposure regions with a predetermined pattern is prepared as shown in (c) of FIG. 1. An exposure method is not limited to means in which a photomask is used, and means such as projection exposure in which an optical system such as a lens or a mirror is used or maskless exposure in which a spatial light modulator, a laser beam or the like is used can be used. The photomask 13 may be provided so as to be in contact with or provided so as not to be in contact with the photosensitive surface treatment agent layer 10.

After that, the photosensitive surface treatment agent layer 10 is irradiated with UV light through the photomask 13 as shown in (c) of FIG. 1. Therefore, the photosensitive surface treatment agent layer 10 is exposed in the exposure regions of the photomask 13.

As a result, thiol generation parts 14 are formed in exposed parts, and a thiol non-generation part 12 is formed in an unexposed part as shown in (d) of FIG. 1.

Examples of the UV light include i rays having a wavelength of 365 nm. In addition, the exposure amount or exposure time does not necessarily need to be large or long enough for deprotection to completely proceed and may be large or long enough for some of thiol groups to be generated.

Next, a catalyst for electroless plating is imparted to the surface to form catalyst layers 15 as shown in (e) of FIG. 1. The catalyst for electroless plating is a catalyst that reduces metallic ions that are included in a plating solution for electroless plating, and examples thereof include silver and palladium.

Thiol groups are exposed on the surface of the thiol generation parts 14. The thiol groups are capable of capturing and reducing the catalyst for electroless plating. Therefore, the catalyst for electroless plating is captured only in the thiol generation parts 14, and the catalyst layers 15 are formed. In addition, as the catalyst for electroless plating, a catalyst that the thiol groups are capable of supporting can be used.

An electroless plating treatment is performed to form plating layers 16 as shown in (f) of FIG. 1. Examples of a material of the plating layer 16 include nickel-phosphorus (NiP) and copper (Cu).

In the present step, the substrate 11 is immersed in an electroless plating bath to reduce the metallic ions on the catalyst surface, and the plating layers 16 are precipitated. At that time, the catalyst layers 15 supporting a sufficient amount of the catalyst are formed on the surfaces of the thiol generation parts 14, which makes it possible to selectively precipitate the plating layers 16 only on the thiol generation parts 14.

A wiring pattern can be formed on a predetermined substrate using the photosensitive surface treatment agent of the present embodiment by the above-described steps.

### Method for Manufacturing Transistor

Furthermore, a method for manufacturing a transistor in which the plating layer 16 obtained in the <method for forming a pattern> is used as a gate electrode will be described using (a) to (d) of FIG. 2.

An insulator layer 17 is formed by a well-known method so as to cover the plating layer 16 and the thiol non-generation parts 12 in an electroless plating pattern formed by the above-described method for forming a pattern as shown in (a) of FIG. 2. The insulator layer 17 may be formed by, for example, using a coating liquid containing one or more resins of an ultraviolet-curable acrylic resin, an epoxy resin, an ene-thiol resin, a silicone resin and the like dissolved in an organic solvent and applying the coating liquid. The insulator layer 17 can be formed in a desired pattern by irradiating the coated film with ultraviolet rays through a mask provided with an opening corresponding to a region where the insulator layer 17 is to be formed. Before the formation of the insulator layer 17, the thiol non-generation parts 12 may be removed as necessary.

A photosensitive surface treatment agent layer 10 is formed on the insulator layer 17 in the same manner as in the above-described method for forming an electroless plating pattern, and thiol generation parts 14 are formed in portions where a source electrode and a drain electrode are to be formed as shown in (b) of FIG. 2.

A catalyst for electroless plating is supported on the thiol generation parts 14 to form catalyst layers 15 in the same manner as in the above-described method for forming an electroless plating pattern, and a plating layer 18 (source electrode) and a plating layer 19 (drain electrode) are formed by performing electroless plating as shown in (c) of FIG. 2. Examples of a material of the plating layers 18 and 19 include nickel-phosphorus (NiP) and copper (Cu), but the plating layers may be formed of a different material from the plating layer 16 (gate electrode) or a different metal such as gold (Au) may be precipitated on the surface of nickel-phosphorus (NiP) or copper (Cu) by performing, for example, electroless gold plating.

A semiconductor layer 21 is formed between the plating layer 18 (source electrode) and the plating layer 19 (drain electrode) as shown in (d) of FIG. 2.

The semiconductor layer 21 may be formed by, for example, producing a solution by dissolving an organic semiconductor material that is soluble in an organic solvent such as TIPS-pentacene (6, 13-bis(triisopropylsilylethynyl)pentacene) in the organic solvent and applying and drying the solution between the plating layer 18 (source electrode) and the plating layer 19 (drain electrode).

In addition, the semiconductor layer 21 may be also formed by adding one or more insulating polymers such as polystyrene (PS) and polymethyl methacrylate (PMMA) to the above-described solution and applying and drying the solution containing the insulating polymers.

When the semiconductor layer 21 is formed as described above, an insulating polymer is formed in a concentrated manner below (on the insulator layer 17 side of) the semiconductor layer 21. In a case where there are polar groups such as thiol groups in the interface between an organic semiconductor and the insulator layer, the transistor performance tends to deteriorate, but the deterioration of the transistor performance can be curbed by providing the organic semiconductor through the above-described insulating polymer in the configuration. A transistor can be manufactured as described above.

According to the above-described method, there is no need to provide a separate chemical resist or the like in the UV exposure step, and the UV exposure step can be made into a simple step where only a photomask is used. Therefore, naturally, there is no need for a step of removing a resist layer. In addition, the catalytic reduction capability of the thiol group also makes it possible to eliminate the necessity of the activation treatment step of the catalyst, which is normally required, and makes high-definition patterning possible while realizing significant cost reduction and time saving. In addition, since the dip coating method can be used, the manufacturing method can be used extremely compatibly even with roll-to-roll steps.

The structure of the transistor is not particularly limited and can be selected as appropriate depending on the purpose. For example, top contact/bottom gate-type, top contact/top gate-type, bottom contact/top gate-type transistors can be manufactured in the same manner.

### <Laminate>

The present embodiment is a laminate containing the photosensitive surface treatment agent of the present embodiment.

The laminate of the present embodiment is a laminate on which a substrate and a metal pattern are laminated together and contains the photosensitive surface treatment agent in unexposed parts in which no patterns are formed.

### <Transistor>

The present embodiment is a transistor containing the photosensitive surface treatment agent of the present embodiment.

The transistor of the present embodiment is a transistor having a laminate on which a substrate and a metal pattern are laminated together and contains the photosensitive surface treatment agent in unexposed parts in which no patterns are formed.

### EXAMPLES

Hereinafter, the present invention will be described more specifically with examples, but the present invention is not limited to the following examples.

### Example 1: Synthesis of Photosensitive Thiol-Generating Monomer

2-((4,5-dimethoxy-2-nitrobenzyl)thio)ethane-1-ol (NBS-OH) represented by the following formula (M) was synthesized by a method to be described below.

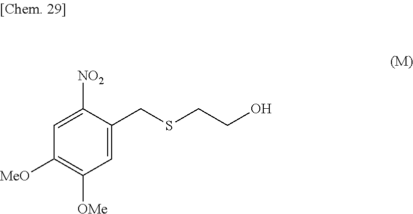 [SMILES: COc1cc(CSCCO)c([N+](=O)[O-])cc1OC]

1-(Bromomethyl)-4,5-dimethoxy-2-nitrobenzene (120.0 g, 435 mmol, 1.0 eq., manufactured by Sigma-Aldrich Co. LLC) and acetonitrile (2.4 L, manufactured by FUJIFILM Wako Pure Chemical Corporation) were added to and stirred in a 5 L four-neck flask under argon.

Cesium carbonate (170.0 g, 0.522 mmol, 1.2 eq., manufactured by FUJIFILM Wako Pure Chemical Corporation) was added thereto, mercaptoethanol (40.8 g, 522 mmol., 1.2 eq., manufactured by Tokyo Chemical Industry Co., Ltd.) was then further added dropwise thereto for 15 minutes, and a reaction was then caused in a bath at 50° C. for 21 hours.

Water (2.4 L) was added thereto, a reaction product was stirred for 10 minutes and then concentrated under reduced pressure (40° C./20 mmHg), and only acetonitrile was distilled away. A residue was moved to a 15 L plastic container, extracted with ethyl acetate (4.8 L) and further extracted from a water layer with ethyl acetate (2.4 L). An ethyl acetate layer was combined therewith, and the reaction product was washed with water (2.4 L) three times and then dried over anhydrous magnesium sulfate. After the desiccant was removed, the reaction product was concentrated under reduced pressure (40° C./20 mmHg), and a brown solid was obtained. Silica gel column purification was performed on an obtained coarse body, and 92.4 g (78.3%) of NBS-OH was obtained.

A measurement result of 1H-NMR (JEOL Ltd., 300 MHz) is shown below.

1H-NMR (300 MHz, CDCl3): δ 2.11 (1H, t), 2.73 (2H, t), 3.76 (2H, m), 3.80 (3H, s), 3.99 (3H, s), 4.15 (2H, s), 6.94 (1H, s), 7.36 (1H, s).

2-((4,5-Dimethoxy-2-nitrobenzyl)thio)ethyl methacrylate (NBS-MEMA) represented by the following formula (11) was synthesized by a method to be described below.

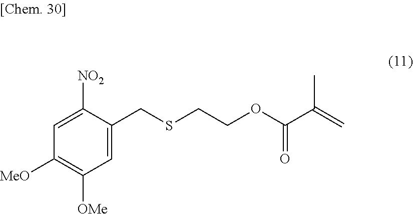 [SMILES: C=C(C)C(=O)OCCSCc1cc(OC)c(OC)cc1[N+](=O)[O-]]

NBS-OH and THF (dry) (400 mL, manufactured by FUJIFILM Wako Pure Chemical Corporation) were added to and dissolved in a 1 L four-neck flask under argon. Triethylamine (11.1 g, 110 mmol, 1.5 eq., manufactured by FUJIFILM Wako Pure Chemical Corporation) was added thereto, the solution was cooled with ice water, methacryloyl chloride (10.7 g, 102 mmol, 1.4 eq., manufactured by FUJIFILM Wako Pure Chemical Corporation) was added dropwise thereto for 30 minutes, and the solution was then stirred overnight (the ice bath was not removed and was left to naturally return to room temperature).

Ice water (400 g) was poured into a reactor, the solution was stirred for five minutes, then, moved to a separatory funnel and extracted with ethyl acetate (800 mL). Next, an ethyl acetate layer was washed with 5% baking soda water (400 mL) twice and with city water (400 mL) three times and then dried over anhydrous sodium sulfate.

After the desiccant was removed, the ethyl acetate layer was concentrated under reduced pressure (40° C./20 mmHg), and 31 g of a yellow solid was obtained. Silica gel column purification was performed on an obtained coarse body, and 16.9 g (67.6%) of NBS-MEMA was obtained.

A measurement result of 1H-NMR (JEOL Ltd., 300 MHz) is shown below.

1H-NMR (300 MHz, CDCl3): δ 1.94 (2H, s), 2.76 (2H, t), 3.95 (3H, s), 4.00 (3H, s), 4.16 (2H, s), 4.31 (2H, t), 5.59 (1H, s), 6.11 (1H, s), 7.27 (1H, s), 7.66 (1H, s)

Poly 2-((4,5-dimethoxy-2-nitrobenzyl)thio)ethyl methacrylate (P-NBS-MEMA) represented by the following formula (12) was synthesized by a method to be described below.

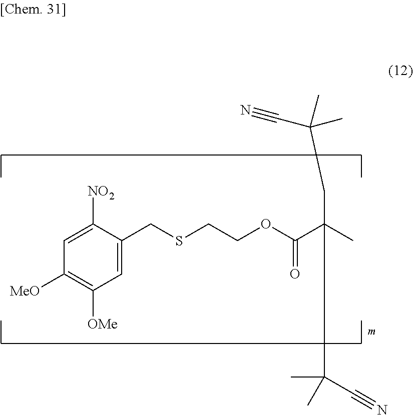

NBS-MEMA (15.0 g, 43.9 mmol, 1.0 eq.) and degassed DMF (30 mL, manufactured by FUJIFILM Wako Pure Chemical Corporation) were added to and dissolved in a 100 mL eggplant flask under argon. AIBN (0.4 g, 2.2 mmol, 0.05 eq., manufactured by FUJIFILM Wako Pure Chemical Corporation) was added thereto, the bath temperature was raised up to 65° C. for 30 minutes, and the solution was heated and stirred at the same temperature for 24 hours.

A reaction solution was naturally cooled, methanol (600 mL) was added dropwise thereto, and the reaction solution was stirred for 30 minutes. A precipitated solid was filtered and washed with methanol (100 mL) three times. The solid was dissolved again in chloroform (150 mL) and added dropwise to methanol (1.5 L) using a dropping funnel for 30 minutes.

After the end of the dropwise addition, the solution was stirred for 15 minutes, then, filtered and washed with methanol (100 mL) three times. An obtained solid was dried under reduced pressure (60° C./<1 mmHg, 16 h), and 13.3 g of intended P-NBS-MEMA was obtained.

Measurement results of 1H-NMR (JEOL Ltd., 300 MHz) and GPC (Tosoh Corporation, HLC-8420GPC, shodex KF-805×2) are shown below.

1H-NMR (300 MHz, CDCl3): δ 0.90-1.37 (m), 1.87-1.94 (m), 2.73 (2H, m), 3.90-4.10 (10H, m), 6.93 (1H, s), 7.56 (1H, s)

GPC: MW: 69770, Mn: 32392, PDI: 2.154

### Example 2

### [Production of Plated Wiring]

A film was formed on a substrate using a surface treatment agent containing the polymer compound (P-NBS-MEMA) represented by the formula (12), and a plated wiring was produced.

Cyclopentanone was added to the polymer compound (P-NBS-MEMA) represented by the formula (12) synthesized in Example 1 to adjust the amount to 0.2 mass %, and a photosensitive surface treatment agent 10a was obtained.

The photosensitive surface treatment agent 10a was applied onto a PEN substrate (manufactured by Teijin Limited, TEONEX Q65HA) with a spin coater (manufactured by Mikasa Co., Ltd., MS-A150, 1000 rpm). After that, the photosensitive surface treatment agent was dried at 100° C. for 10 minutes, and a photosensitive surface treatment agent layer was formed.

Next, the substrate on which the photosensitive surface treatment agent layer was fully formed was exposed to light having a wavelength of 365 nm at 1000 mJ/cm² through a photomask to make the photosensitive surface treatment agent layer exposed, thereby forming thiol generation parts in exposed parts and thiol non-generation parts in unexposed parts.

Next, the substrate was immersed in a catalyst colloid solution for electroless plating (MELPLATE ACTIVATOR 7331, manufactured by Meltex Inc.) at room temperature for 10 minutes to attach a catalyst (Pd) to the thiol generation parts. After the surface was washed with water, the substrate was immersed in an electroless plating solution (MELPLATE NI-867, manufactured by Meltex Inc.) at 73° C. for one minute, and nickel phosphorus was precipitated on the catalyst to produce a fine plated wiring.

### [Evaluation of Plated Wiring and Solubility]

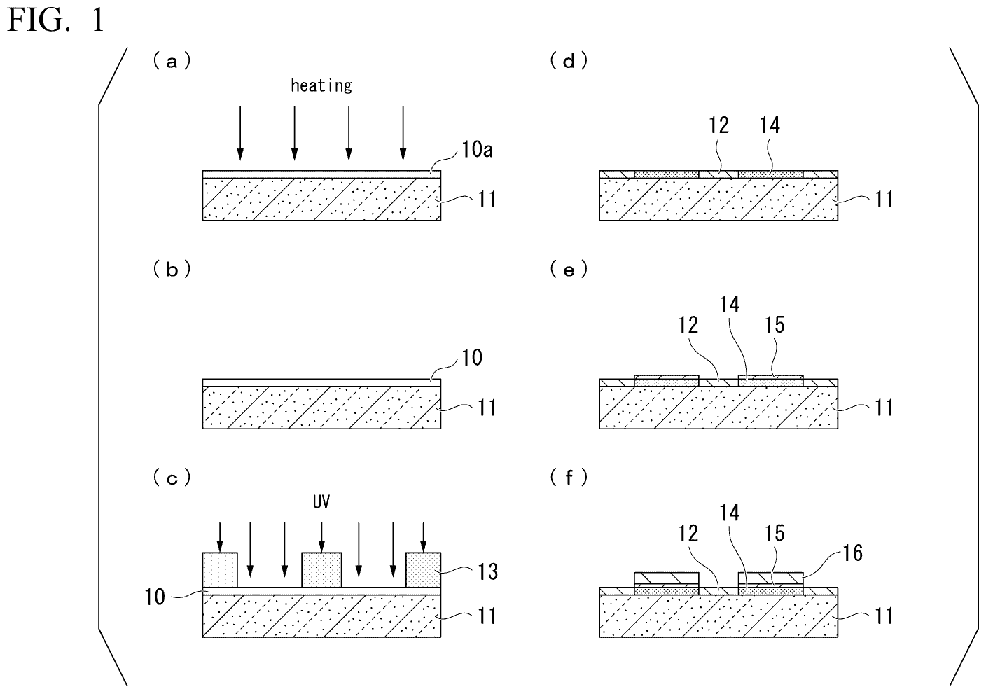

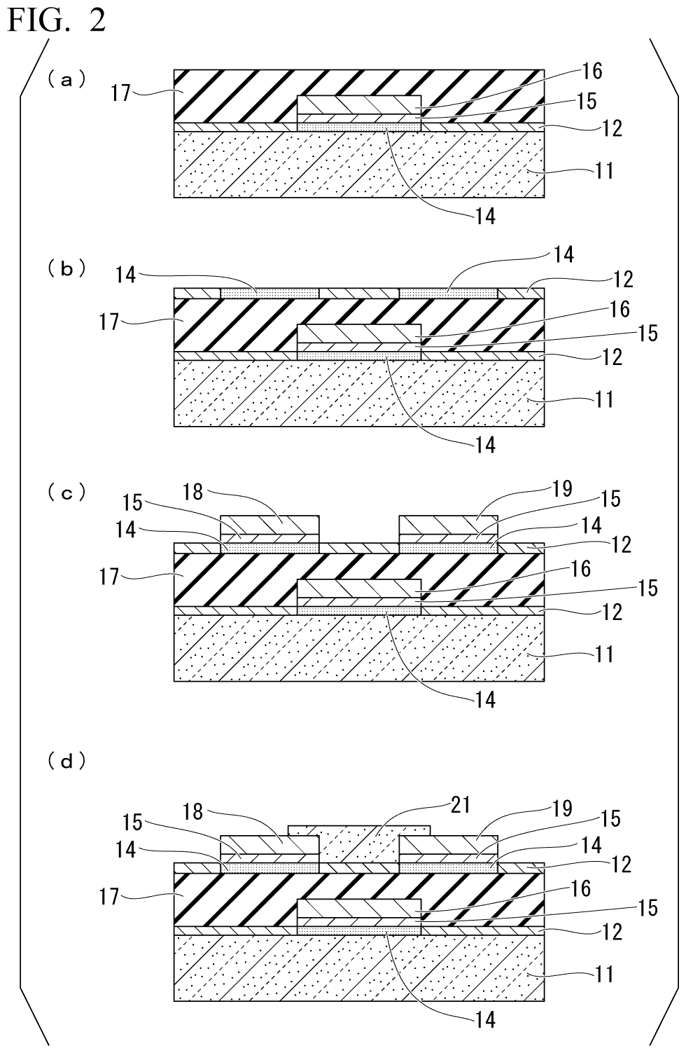

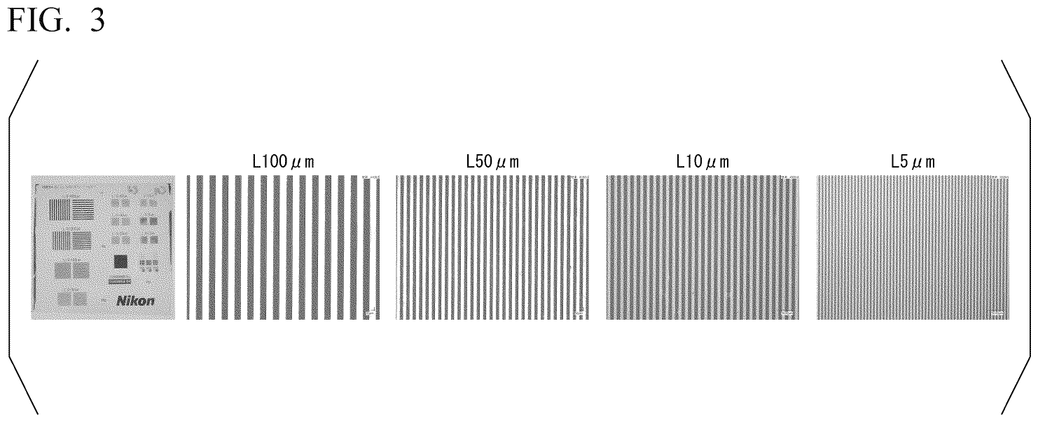

FIG. 3 shows an overall photograph and optical microscope (manufactured by Keyence Corporation, VHX-7000) images of the PEN substrate on which a plated wiring treatment was performed in the example.

From FIG. 3, it was possible to confirm visually and with a microscope that high-definition favorable plated wirings were formed even in fine parts with no use of a resist even in the low-temperature process of 100° C. or lower. In addition, neither peeling nor dissolution was shown in each layer.

Furthermore, the polymer compound (P-NBS-MEMA) represented by the formula (12) synthesized in Example 1 easily dissolved in cyclopentanone and no temporal changes were shown.

The above-described results show that, according to the photosensitive surface treatment agent 10a, it was possible to selectively pattern thiol at an arbitrary position only with light irradiation, and sufficient adhesion could be obtained even after the lamination of metal films using plating. Simplification of management, cost reduction or the like can be expected in the manufacturing or transportation of films for wiring formation and the manufacturing steps of electronic materials or the like in which the films for wiring formation are used.

In addition, it was found that high-definition electroless plating wirings can be provided to smooth substrates with no use of a resist even in low-temperature processes. Therefore, it is possible to eliminate a heating step in each step, for example, a surface treatment, resist development or a resist peeling step where a large number of medicines are used or PEB where energy is required, and effects can be expected in terms of the economic aspect and the environmental preservation aspect. In addition, since light having a wavelength (365 nm) that cause a smaller load on substrates than DUV can be applied, the present invention is a suitable technique for processes on films that are expected to be applied to roll to roll (R to R).

Furthermore, the use of the present example can be expected to make it possible to produce transparent electrodes at a low cost. When the formation of a multilayer wiring such as an electronic device where this is repeatedly performed is taken into account, the effect of a resist-free process is extremely large. In addition, if the reduction of the amount of a chemical substance used and a short-stage device manufacturing process can be realized by the present invention, not only an effect of improving mass production and economic efficiency but also the reduction of the load relating to the development and maintenance of manufacturing devices can be achieved.

In addition, organic thin films that are brought by the present invention function even as extremely thin films of approximately 2 to 10 nm and the environmental load is extremely small.

### Example 3

(Synthesis of 4,5-dimethoxy-2-nitrobenzaldehyde)

3,4-Dimethoxybenzaldehyde (50.1 g, 301 mmol) was put into a 500 mL eggplant flask and dissolved in acetic acid (120 mL), fuming nitric acid (57 mL, 930 mmol) was slowly added dropwise thereto on an ice bath, and the solution was stirred at 0° C. for two hours.

A reaction solution was poured into cold water (800 mL), suction-filtered and sequentially washed with pure water and hexane. The reaction solution was recrystallized (ethanol) by a slow cooling method, and 35.2 g (166 mmol, 55%) of a yellow crystal was obtained.

A measurement result of 1H-NMR (JEOL Ltd, 400 MHZ) and a reaction formula are shown below.

¹H-NMR (CDCl₃/TMS, 400 MHZ): δ 4.03 (3H, s), 4.04 (3H, s), 7.43 (1H, s), 7.62 (1H, s), 10.5 (1H, s).

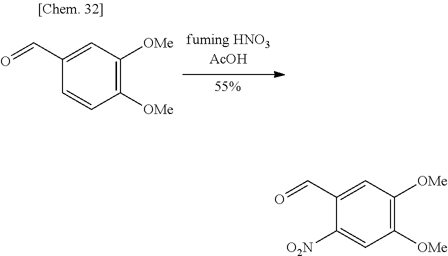 [SMILES: COc1ccc(C=O)cc1OC ; COc1cc(C=O)c([N+](=O)[O-])cc1OC]

(Synthesis of 4,5-dimethoxy-2-nitrobenzyl Alcohol)

4,5-Dimethoxy-2-nitrobenzaldehyde (14.1 g, 66.8 mmol) was put into a 500 mL eggplant flask and dissolved in tetrahydrofuran (200 mL) and methanol (100 mL), a small amount of sodium borohydride (3.79 g, 100 mmol) was added to each solution on an ice bath, and the solution was then stirred at 0° C. for 30 minutes and further stirred at room temperature for 90 minutes.

After concentration, an organic layer was extracted by adding ethyl acetate (100 mL×3), pure water (200 mL) and 2N hydrochloric acid (25 mL), washed with saturated salt water (200 mL×2), dried over anhydrous magnesium sulfate, filtered and concentrated, thereby obtaining 13.8 g (64.7 mmol, 97%) of a yellow solid.

A measurement result of 1H-NMR (JEOL Ltd, 400 MHz) and a reaction formula are shown below.

¹H-NMR (CDCl₃/TMS, 400 MHZ): δ 2.61 (1H, br s), 3.97 (3H, s), 4.01 (3H, s), 4.97 (2H, s), 7.18 (1H, s), 7.72 (1H, s).

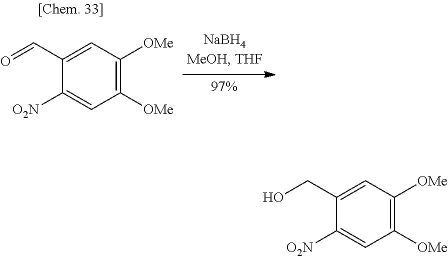 [SMILES: COc1cc(C=O)c([N+](=O)[O-])cc1OC ; COc1cc(CO)c([N+](=O)[O-])cc1OC]

(Synthesis of (4,5-dimethoxy-2-nitrophenyl)Methyl(2,5-dioxo-1-pyrrolidinyl)Carbonate)

4,5-Dimethoxy-2-nitrobenzyl alcohol (1.00 g, 4.69 mmol) was put into a 100 mL eggplant flask and dissolved in dry acetonitrile (25 mL), di(N-succinimidyl) carbonate (2.40 g, 9.37 mmol) and triethylamine (2.0 mL, 14.4 mmol) were added thereto, and the solution was stirred at room temperature for 18 hours under a nitrogen atmosphere.

After concentration, an organic layer was extracted by adding ethyl acetate (60 mL×4), pure water (60 mL) and 2N hydrochloric acid (10 mL), washed with saturated salt water (200 mL), dried over anhydrous magnesium sulfate, filtered and concentrated. The organic layer was purified by silica gel column chromatography (hexane:ethyl acetate=1:2), thereby obtaining 1.89 g (3.55 mmol, 76%) of a yellow powder.

A measurement result of 1H-NMR (JEOL Ltd, 400 MHz) and a reaction formula are shown below.

¹H-NMR (CDCl₃/TMS, 400 MHZ): 8 2.86 (4H, s), 3.98 (3H, s), 4.07 (3H, s), 5.80 (2H, s), 7.05 (1H, s), 7.78 (1H, s).

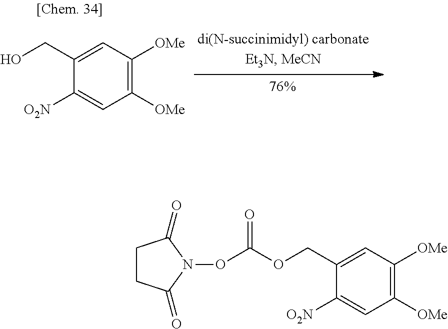 [SMILES: COc1cc(CO)c([N+](=O)[O-])cc1OC ; COc1cc(COC(=O)ON2C(=O)CCC2=O)c([N+](=O)[O-])cc1OC]

(Synthesis of S-octyl O-[(4,5-dimethoxy-2-nitrophenyl)Methyl]Carbonate)

(4,5-Dimethoxy-2-nitrophenyl)methyl (2,5-dioxo-1-pyrrolidinyl) carbonate (0.10 g, 0.28 mmol) was put into a 10 mL two-neck test tube and dissolved in dry tetrahydrofuran (4 mL), 4-dimethylaminopyridine (DMAP) (0.14 g, 1.14 mmol) and 1-octanethiol (0.19 mL, 1.12 mmol) were added thereto, and the solution was stirred at room temperature for 24 hours under a nitrogen atmosphere.

After concentration, an organic layer was extracted by adding ethyl acetate (50 mL×3), pure water (50 mL) and 2N hydrochloric acid (3 mL), washed with saturated salt water (200 mL), dried over anhydrous magnesium sulfate, filtered and concentrated. The organic layer was purified by silica gel column chromatography (hexane:ethyl acetate=4:1), thereby obtaining 0.06 g (0.15 mmol, 52%) of a yellow solid.

A measurement result of 1H-NMR (JEOL Ltd, 400 MHZ) and a reaction formula are shown below.

¹H-NMR (CDCl₃/TMS, 400 MHz): δ 0.88 (3H, t, J=6.9 Hz), 1.19-1.44 (10H, m), 1.66 (2H, quint, J=7.5 Hz), 2.90 (2H, t, J=7.4 Hz), 3.96 (3H, s), 3.99 (3H, s), 5.67 (2H, s), 7.01 (1H, s), 7.74 (1H, s).

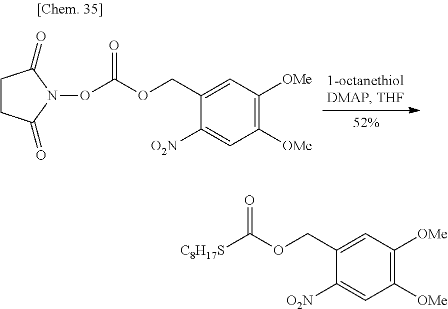

### Example 4

(Synthesis of 4,5-dimethoxy-2-nitrobenzyl Bromide)

4,5-Dimethoxy-2-nitrobenzyl alcohol (0.50 g, 2.34 mmol) was put into a 30 mL two-neck eggplant flask and dissolved in dry tetrahydrofuran (10 mL), triphenyl phosphine (0.927 g, 3.53 mmol) and carbon tetrabromide (1.16 g, 3.50 mmol) were added thereto, and the solution was stirred at room temperature for one hour under a nitrogen atmosphere.

After filtration, a filtrate was concentrated. The filtrate was purified by silica gel column chromatography (hexane:ethyl acetate=3:1), thereby obtaining 0.48 g (1.74 mmol, 74%) of a yellow powder.

A measurement result of 1H-NMR (JEOL Ltd, 400 MHz) and a reaction formula are shown below.

¹H-NMR (CDCl₃/TMS, 400 MHz): δ 3.97 (3H, s), 4.00 (3H, s), 4.88 (2H, s), 6.95 (1H, s), 7.68 (1H, s).

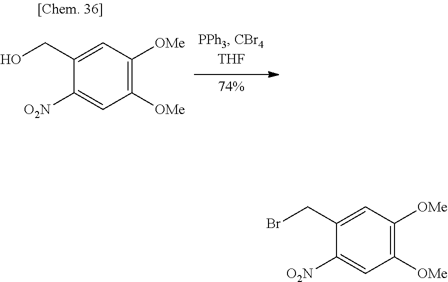 [SMILES: COc1cc(CO)c([N+](=O)[O-])cc1OC ; COc1cc(CBr)c([N+](=O)[O-])cc1OC]

(Synthesis of 4,5-dimethyoxy-2-nitrobenzyl Octyl Sulfide)

Dry acetonitrile (30 mL), potassium carbonate (0.57 g, 4.14 mmol) and 1-octanethiol (0.61 g, 4.14 mmol) were put into a 100 mL two-neck eggplant flask and stirred at room temperature for two hours under a nitrogen atmosphere. 4,5-Dimethoxy-2-nitrobenzyl bromide (0.80 g, 2.90 mmol) was added thereto and refluxed for five hours under a nitrogen atmosphere.

After concentration, an organic layer was extracted by adding chloroform (70 mL×3), pure water (100 mL) and 2N hydrochloric acid (4 mL), washed with saturated salt water (200 mL), dried over anhydrous magnesium sulfate, filtered and concentrated. The organic layer was purified by silica gel column chromatography (hexane:ethyl acetate=6:1), thereby obtaining 0.56 g (1.65 mmol, 56%) of a yellow powder.

A measurement result of 1H-NMR (JEOL Ltd, 400 MHz) and a reaction formula are shown below.

¹H-NMR (CDCl₃/TMS, 400 MHZ): δ 0.88 (3H, t, J=6.9 Hz), 1.19-1.44 (10H, m), 1.49-1.61 (2H, m), 2.49 (2H, t, J=7.5 Hz), 3.94 (3H, s), 3.98 (3H, s), 4.10 (2H, s), 6.96 (1H, s), 7.62 (1H, s).

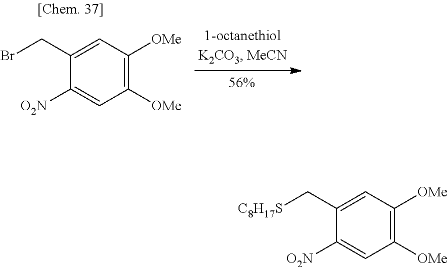

### Example 5

(Synthesis of 1-(4,5-dimethoxy-2-nitrophenyl)Ethanone)

3,4-Dimethoxyacetophenone (50.1 g, 278 mmol) was put into a 500 mL eggplant flask and dissolved in acetic acid (200 mL), fuming nitric acid (47.6 mL, 1128 mmol) was slowly added dropwise thereto on an ice bath, and the solution was stirred at 0° C. for 90 minutes. A reaction solution was poured into cold water (1500 mL), suction-filtered and sequentially washed with pure water and hexane. The reaction solution was recrystallized (ethanol) by a slow cooling method, and 42.3 g (188 mmol, 68%) of a yellow crystal was obtained.

A measurement result of 1H-NMR (JEOL Ltd, 400 MHz) and a reaction formula are shown below.

¹H-NMR (CDCl₃/TMS, 400 MHz): δ 2.51 (3H, s), 3.99 (3H, s), 4.02 (3H, s), 6.76 (1H, s), 7.62 (1H, s).

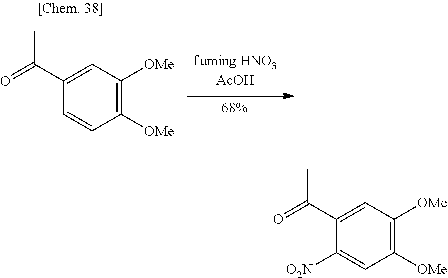 [SMILES: COc1ccc(C(C)=O)cc1OC ; COc1cc(C(C)=O)c([N+](=O)[O-])cc1OC]

(Synthesis of 1-(4,5-dimethoxy-2-nitrophenyl)Ethanol)

1-(4,5-dimethoxy-2-nitrophenyl)ethanone (8.78 g, 39.0 mmol) was put into a 300 mL eggplant flask and dissolved in tetrahydrofuran (100 mL) and methanol (50 mL), a small amount of sodium borohydride (2.06 g, 58.5 mmol) was added to each solution on an ice bath, and the solution was then stirred at 0° C. for 30 minutes and further stirred at room temperature for two hours.

After concentration, an organic layer was extracted by adding dichloromethane (100 mL×3), pure water (100 mL) and 2N hydrochloric acid (30 mL), washed with saturated salt water (100 mL), dried over anhydrous magnesium sulfate, filtered and concentrated, thereby obtaining 8.28 g (36.4 mmol, 96%) of a yellow solid.

A measurement result of 1H-NMR (JEOL Ltd, 400 MHz) and a reaction formula are shown below.

¹H-NMR (CDCl₃/TMS, 400 MHZ): δ 1.57 (3H, d, J=6.3 Hz), 2.26 (1H, d, J=3.7 Hz), 3.95 (3H, s), 4.01 (3H, s), 4.97 (1H, qd, J=6.3, 3.7 Hz), 7.31 (1H, s), 7.58 (1H,s).

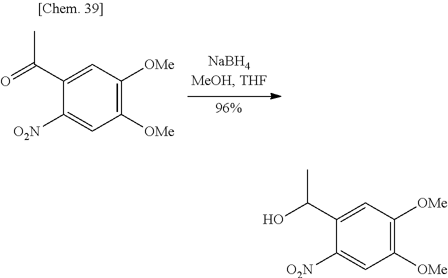 [SMILES: COc1cc(C(C)=O)c([N+](=O)[O-])cc1OC ; COc1cc(C(C)O)c([N+](=O)[O-])cc1OC]

(Synthesis of 1-(4,5-dimethoxy-2-nitrophenyl)Ethyl (2,5-dioxo-1-pyrrolidinyl)Carbonate)

1-(4,5-Dimethoxy-2-nitrophenyl)ethanol (1.00 g, 4.43 mmol) was put into a 100 mL two-neck eggplant flask and dissolved in dry acetonitrile (15 mL), di(N-succinimidyl) carbonate (1.71 g, 6.68 mmol) and triethylamine (1.8 mL, 13.0 mmol) were added thereto, and the solution was stirred at room temperature for 18 hours under a nitrogen atmosphere.

After concentration, an organic layer was extracted by adding chloroform (80 mL×4), pure water (100 mL) and 2N hydrochloric acid (10 mL), washed with saturated salt water (200 mL), dried over anhydrous magnesium sulfate, filtered and concentrated. The organic layer was purified by silica gel column chromatography (hexane:ethyl acetate=1:1), thereby obtaining 0.69 g (1.87 mmol, 43%) of a yellow powder.

A measurement result of 1H-NMR (JEOL Ltd, 400 MHZ) and a reaction formula are shown below.

¹H-NMR (CDCl₃/TMS, 400 MHZ): δ 1.77 (3H, d, J=6.4 Hz), 2.80 (4H, s), 3.96 (3H, s), 4.07 (3H, s), 6.51 (1H, q, J=6.4 Hz), 7.08 (1H, s), 7.65 (1H, s).

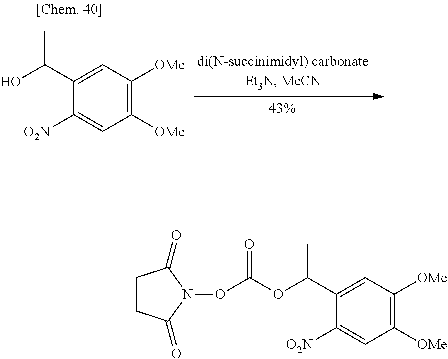 [SMILES: COc1cc(C(C)O)c([N+](=O)[O-])cc1OC ; COc1cc(C(C)OC(=O)ON2C(=O)CCC2=O)c([N+](=O)[O-])cc1OC]

(Synthesis of S-octyl O-[1-(4,5-dimethoxy-2-nitrophenyl)Ethyl]Carbonate))

1-(4,5-Dimethoxy-2-nitrophenyl)ethyl (2,5-dioxo-1-pyrrolidinyl) carbonate (0.37 g, 1.00 mmol) was put into a 20 mL two-neck eggplant flask and dissolved in dry tetrahydrofuran (10 mL), 4-dimethylaminopyridine (DMAP) (0.25 g, 4.01 mmol) and 1-octanethiol (0.68 mL, 3.92 mmol) were added thereto, and the solution was stirred at room temperature for 24 hours under a nitrogen atmosphere.

After concentration, an organic layer was extracted by adding chloroform (20 mL×3), pure water (50 mL) and 2N hydrochloric acid (5 mL), washed with saturated salt water (100 mL), dried over anhydrous magnesium sulfate, filtered and concentrated. The organic layer was purified by silica gel column chromatography (hexane:ethyl acetate=8:1), thereby obtaining 0.18 g (0.45 mmol, 45%) of a yellow solid.

A measurement result of 1H-NMR (JEOL Ltd, 400 MHz) and a reaction formula are shown below.

¹H-NMR (CDCl₃/TMS, 400 MHZ): δ 0.87 (3H, t, J=6.9 Hz), 1.27-1.39 (10H, m), 1.43-1.62 (2H, m), 1.65 (3H, d, J=6.4 Hz), 2.74-2.87 (2H, m), 3.94 (3H, s), 3.99 (3H, s), 6.58 (1H, q, J=6.4 Hz), 7.02 (1H, s), 7.61 (1H, s).

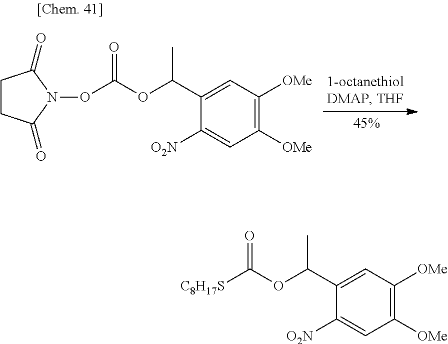

### Example 6

(Synthesis of S-octyl O-[1-(4,5-dimethoxy-2-nitrophenyl)Ethyl Bromide)

1-(4,5-Dimethoxy-2-nitrophenyl)ethanol (10.1 g, 44.4 mmol) was put into a 200 mL two-neck eggplant flask and dissolved in dry dichloromethane (300 mL), phosphorus tribromide (15.2 g, 56.2 mmol) dissolved in dry dichloromethane (50 mL) was slowly added dropwise thereto under an ice bath, and the solution was stirred at 0° C. for one hour.

An organic layer was collected by adding pure water (150 mL), a water layer was extracted with methylene chloride (100 mL×2), the organic layer was combined therewith, the layers were washed with saturated salt water (150 mL), dried over anhydrous magnesium sulfate, filtered and concentrated. The layers were purified by silica gel column chromatography (hexane:ethyl acetate=4:1), thereby obtaining 8.94 g (30.8 mmol, 70%) of a yellow viscous body.

A measurement result of 1H-NMR (JEOL Ltd, 400 MHz) and a reaction formula are shown below.

¹H-NMR (CDCl₃/TMS, 400 MHZ): δ 2.07 (3H, d, J=6.9 Hz), 3.95 (3H, s), 4.02 (3H, s), 6.04 (1H, q, J=6.9 Hz), 7.28 (1H, s), 7.46 (1H, s).

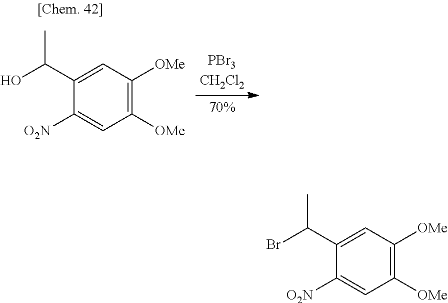 [SMILES: COc1cc(C(C)O)c([N+](=O)[O-])cc1OC ; COc1cc(C(C)Br)c([N+](=O)[O-])cc1OC]

(Synthesis of 1-(4,5-dimethoxy-2-nitrophenyl)Ethyl Octyl Sulfide)

Dry acetonitrile (30 mL), potassium carbonate (0.52 g, 4.07 mmol) and 1-octanethiol (0.71 mL, 4.07 mmol) were put into a 100 mL two-neck eggplant flask and stirred at room temperature for two hours under a nitrogen atmosphere. 1-(4,5-Dimethoxy-2-nitrophenyl) ethyl bromide (0.84 g, 2.91 mmol) was added thereto and refluxed for four hours under a nitrogen atmosphere.

After concentration, an organic layer was extracted by adding chloroform (70 mL×3), pure water (100 mL) and 2N hydrochloric acid (4 mL), washed with saturated salt water (200 mL), dried over anhydrous magnesium sulfate, filtered and concentrated. The organic layer was purified by silica gel column chromatography (hexane:ethyl acetate=8:1), thereby obtaining 0.58 g (1.65 mmol, 57%) of a yellow powder.

A measurement result of 1H-NMR (JEOL Ltd, 400 MHz) and a reaction formula are shown below.

¹H-NMR (CDCl₃/TMS, 400 MHZ): δ 0.87 (3H, t, J=6.9 Hz), 1.14-1.37 (10H, m), 1.39-1.51 (2H, m), 1.57 (3H, d, J=7.0 Hz), 2.21-2.32 (1H, m), 2.32-2.43 (1H, m), 3.94 (3H, s), 3.99 (3H, s), 4.85 (1H, q, J=7.0 Hz), 7.35 (1H, s), 7.41 (1H, s).

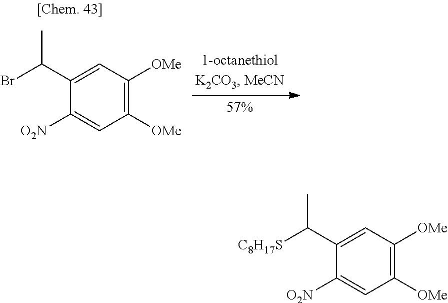

### Example 7

(Synthesis of 1-(3,4-dimethoxyphenyl)-2-methyl-1-propanone)

1,2-Dimethoxybenzene (64.8 mL, 50.8 mmol) and isobutyric anhydride (93.0 mL, 576 mmol) were put into a 500 mL eggplant flask, iodine (7.72 g, 30.4 mmol) was added thereto, and the components were stirred at room temperature for two and a half hours. After the components were washed with a saturated sodium thiosulfate aqueous solution (50 mL), an organic layer was extracted by adding ethyl acetate (100 mL×3) and pure water (150 mL), washed with saturated salt water (100 mL), dried over anhydrous magnesium sulfate, filtered and concentrated, thereby obtaining 73.1 g (351 mmol, 69%) of a brown viscous body.

A measurement result of 1H-NMR (JEOL Ltd, 400 MHz) and a reaction formula are shown below.

¹H-NMR (CDCl₃/TMS, 400 MHZ): δ 1.22 (6H, d, J=6.9 Hz), 3.56 (1H, sept, J =6.9 Hz), 3.94 (3H, s), 3.95 (3H, s), 6.90 (1H, d, J=8.5 Hz), 7.55 (1H, d, J=2.0 Hz), 7.60 (1H, dd, J=8.5, 2.0 Hz).

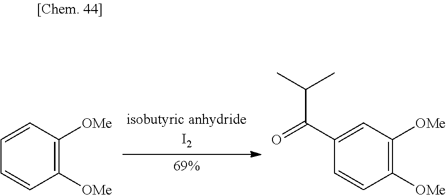

(Synthesis of 1-(4,5-dimethoxy-2-nitrophenyl)-2-methyl-1-propanone)

1-(3,4-Dimethoxyphenyl)-2-methyl-1-propanone (13.6 g, 65.1 mmol) was put into a 200 mL eggplant flask and dissolved in acetic acid (32.5 mL), fuming nitric acid (10.8 mL, 260 mmol) was slowly added dropwise thereto on an ice bath, and the solution was stirred at 0° C. for two hours.

A reaction solution was poured into cold water (800 mL), suction-filtered and sequentially washed with pure water and hexane. The reaction solution was recrystallized (ethanol) by a slow cooling method, and 9.56 g (37.8 mmol, 58%) of a yellow crystal was obtained.

A measurement result of 1H-NMR (JEOL Ltd, 400 MHz) and a reaction formula are shown below.

¹H-NMR (CDCl₃/TMS, 400 MHZ): δ 1.22 (6H, d, J=7.0 Hz), 2.91 (1H, sept, J =7.0 Hz), 3.98 (3H, s), 3.99 (3H, s), 6.69 (1H, s), 7.66 (1H, s).

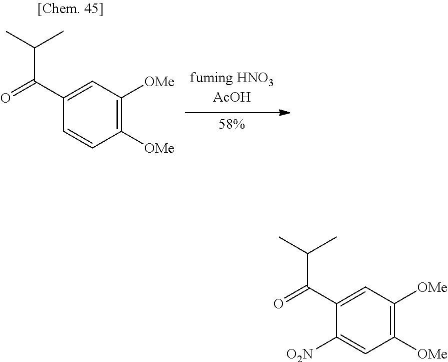 [SMILES: COc1ccc(C(=O)C(C)C)cc1OC ; COc1cc(C(=O)C(C)C)c([N+](=O)[O-])cc1OC]

(Synthesis of 1-(4,5-dimethoxy-2-nitrophenyl)-2-methylpropanone)

1-(4,5-Dimethoxy-2-nitrophenyl)-2-methyl-1-propanone (10.2 g, 40.4 mmol) was put into a 300 mL eggplant flask and dissolved in tetrahydrofuran (80 mL) and methanol (40 mL), a small amount of sodium borohydride (2.32 g, 61.3 mmol) was added to each solution on an ice bath, and the solution was then stirred at 0° C. for 30 minutes and further stirred at room temperature for two hours.

After concentration, an organic layer was extracted by adding dichloromethane (100 mL×3), pure water (100 mL) and 2N hydrochloric acid (35 mL), washed with saturated salt water (150 mL), dried over anhydrous magnesium sulfate, filtered and concentrated, thereby obtaining 10.2 g (40.0 mmol, 99%) of a yellow solid.

A measurement result of 1H-NMR (JEOL Ltd, 400 MHz) and a reaction formula are shown below.

¹H-NMR (CDCl₃/TMS, 400 MHz): δ 0.96 (6H, d, J=6.8 Hz), 1.97-2.11 (1H, m), 2.18 (1H, br s), 3.95 (3H, s), 3.99 (3H, s), 5.27 (1H, br d, J=3.6 Hz), 7.21 (1H, s), 7.57 (1H, s).

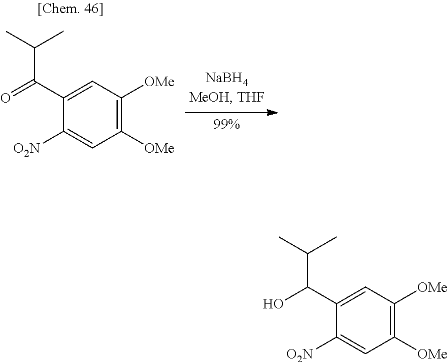 [SMILES: COc1cc(C(=O)C(C)C)c([N+](=O)[O-])cc1OC ; COc1cc(C(O)C(C)C)c([N+](=O)[O-])cc1OC]

(Synthesis of 1-(4,5-dimethoxy-2-nitrophenyl)-2-methylpropyl (2,5-dioxo-1-pyrrolidinyl)Carbonate)

1-(4,5-Dimethoxy-2-nitrophenyl)-2-methylpropanone (4.3 g, 16.8 mmol) was put into a 50 mL two-neck eggplant flask and dissolved in dry acetonitrile (30 mL), di(N-succinimidyl) carbonate (8.92 g, 34.8 mmol) and triethylamine (4.7 mL, 33.9 mmol) were added thereto, and the solution was stirred at room temperature for 18 hours under a nitrogen atmosphere.

After concentration, an organic layer was extracted by adding chloroform (50 mL×4), pure water (50 mL) and 2N hydrochloric acid (10 mL), washed with saturated salt water (200 mL), dried over anhydrous magnesium sulfate, filtered and concentrated. The organic layer was purified by silica gel column chromatography (hexane:ethyl acetate=2:1), thereby obtaining 0.52 g (4.24 mmol, 64%) of a yellow powder.

A measurement result of 1H-NMR (JEOL Ltd, 400 MHz) and a reaction formula are shown below.

¹H-NMR (CDCl₃/TMS, 400 MHZ): δ 1.04 (3H, d, J=6.9 Hz), 1.10 (3H, d, J=6.9 Hz), 2.23-2.31 (1H, m), 2.79 (4H, s), 3.95 (3H, s), 4.06 (3H, s), 6.41 (1H, d, J=5.0 Hz), 6.97 (1H, s), 7.67 (1H, s).

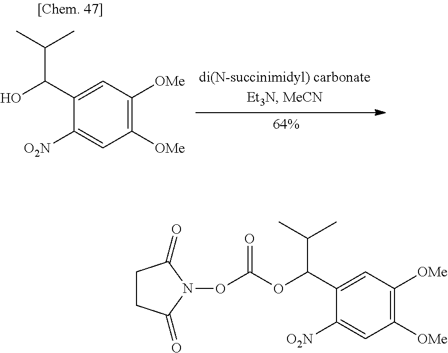 [SMILES: COc1cc(C(O)C(C)C)c([N+](=O)[O-])cc1OC ; COc1cc(C(OC(=O)ON2C(=O)CCC2=O)C(C)C)c([N+](=O)[O-])cc1OC]

(Synthesis of S-octyl O-(1(4,5-dimethoxy-2-nitrophenyl)-2-methylpropyl)Carbonate)

1-(4,5-Dimethoxy-2-nitrophenyl)-2-methylpropyl (2,5-dioxo-1-pyrrolidinyl) carbonate (98.5 mg, 0.25 mmol) was put into a 10 mL two-neck test tube and dissolved in dry tetrahydrofuran (2 mL), 4-dimethylaminopyridine (DMAP) (64.3 mg, 0.53 mmol) and 1-octanethiol (0.087 mL, 0.50 mmol) were added thereto, and the solution was stirred at room temperature for 24 hours under a nitrogen atmosphere.

After concentration, an organic layer was extracted by adding ethyl acetate (60 mL×3), pure water (60 mL) and 2N hydrochloric acid (2 mL), washed with saturated salt water (100 mL), dried over anhydrous magnesium sulfate, filtered and concentrated. The organic layer was purified by silica gel column chromatography (hexane:ethyl acetate=4:1), thereby obtaining 43.6 mg (0.10 mmol, 41%) of a yellow solid.

A measurement result of 1H-NMR (JEOL Ltd, 400 MHz) and a reaction formula are shown below.

¹H-NMR (CDCl₃/TMS, 400 MHZ): δ 0.87 (3H, t, J=6.6 Hz), 1.00 (3H, d, J=6.9 Hz), 1.02 (3H, d, J=6.9 Hz), 1.18-1.39 (10H, m), 1.53-1.63 (2H, m), 2.12-2.24 (1H, m), 2.74-2.86 (2H, m), 3.95 (3H, s), 3.97 (3H, s), 6.47 (2H, d, J=5.5 Hz), 6.92 (1H, s), 7.63 (1H, s).

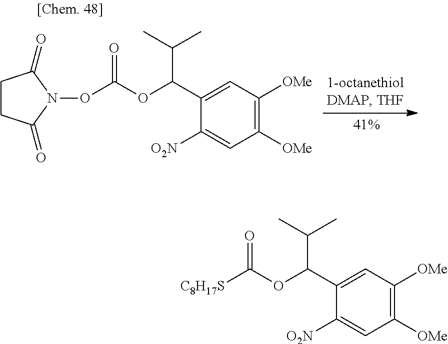

### Example 8

(Synthesis of 1-(4,5-dimethoxy-2-nitrophenyl)-2-methylpropyl Bromide)

1-(4,5-Dimethoxy-2-nitrophenyl)-2-methylpropanone (3.17 g, 12.4 mmol) was put into a 500 mL two-neck eggplant flask and dissolved in dry benzene (250 mL), pyridine (0.5 mL, 6.20 mmol) was added thereto, phosphorus tribromide (1.80 mL, 19.0 mmol) dissolved in dry benzene (100 mL) was slowly added dropwise thereto under an ice bath, and the solution was stirred at 0° C. for two hours.

After the solution was concentrated by adding pure water (10 mL), an organic layer was extracted by adding ethyl acetate (100 mL×3) and pure water (150 mL), washed with saturated salt water (150 mL), dried over anhydrous magnesium sulfate, filtered and concentrated. The organic layer was purified by silica gel column chromatography (hexane:ethyl acetate=5:1), thereby obtaining 2.05 g (6.44 mmol, 52%) of a yellow viscous body.

A measurement result of 1H-NMR (JEOL Ltd, 400 MHZ) and a reaction formula are shown below.

¹H-NMR (CDCl₃/TMS, 400 MHZ): δ 0.94 (3H, d, J=6.8 Hz), 1.22 (3H, d, J=6.6 Hz), 2.23-2.35 (1H, m), 3.95 (3H, s), 3.99 (3H, s), 5.73 (1H, d, J=8.0 Hz), 7.22 (1H, s), 7.45 (1H, s).

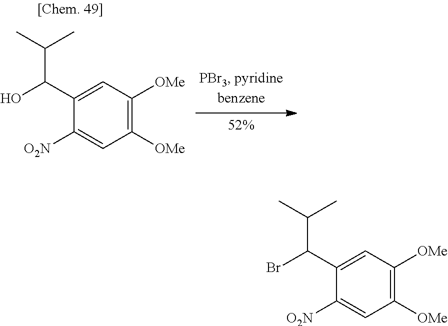 [SMILES: COc1cc(C(O)C(C)C)c([N+](=O)[O-])cc1OC ; COc1cc(C(Br)C(C)C)c([N+](=O)[O-])cc1OC]

(Synthesis of 1-(4,5-dimethoxy-2-nitrophenyl)-2-methylpropyl Octyl Sulfide)

Dry acetonitrile (10 mL), potassium carbonate (73.0 mg, 0.53 mmol) and 1-octanethiol (0.10 mL, 0.58 mmol) were put into a 50 mL two-neck eggplant flask and stirred at room temperature for two hours under a nitrogen atmosphere. 1-(4,5-Dimethoxy-2-nitrophenyl)-2-methylpropyl bromide (0.11 g, 0.35 mmol) was added thereto and refluxed for four hours under a nitrogen atmosphere.

After concentration, an organic layer was extracted by adding chloroform (30 mL×3), pure water (100 mL) and 2N hydrochloric acid (2 mL), washed with saturated salt water (100 mL), dried over anhydrous magnesium sulfate, filtered and concentrated. The organic layer was purified by silica gel column chromatography (hexane:ethyl acetate=8:1), thereby obtaining 12.5 mg (0.03 mmol, 9%) of a yellow powder.

A measurement result of 1H-NMR (JEOL Ltd, 400 MHz) and a reaction formula are shown below.

¹H-NMR (CDCl₃/TMS, 400 MHZ): δ 0.87 (3H, t, J=6.7 Hz), 0.89 (3H, d, J=6.6 Hz), 1.11 (3H, d, J=6.7 Hz), 1.14-1.36 (10H, m), 1.39-1.50 (2H, m), 1.94-2.07 (1H, m), 2.16-2.26 (1H, m), 2.29-2.40 (1H, m), 3.94 (3H, s), 3.98 (3H, s), 4.60 (1H, d, J=8.1 Hz), 7.32 (1H, s), 7.38 (1H, s).

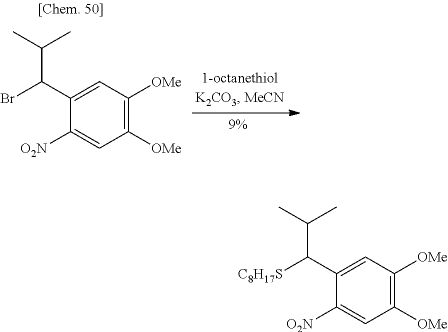

### Example 9

(Synthesis of 1-(3,4-dimethoxyphenyl)-2,2-dimethyl-1-propanone)

1,2-Dimethoxybenzene (27.7 mL, 217 mmol) and pivalic anhydride (44.0 mL, 217 mmol) were put into a 200 mL eggplant flask, iodine (3.30 g, 13.0 mmol) was added thereto, and the solution was stirred at room temperature for 20 hours. After the solution was washed with a saturated sodium thiosulfate aqueous solution (100 mL), an organic layer was extracted by adding ethyl acetate (100 mL×3) and pure water (150 mL), washed with saturated salt water (150 mL×2), dried over anhydrous magnesium sulfate, filtered and concentrated, thereby obtaining 31.5 g (142 mmol, 65%) of a brown viscous body.

A measurement result of 1H-NMR (JEOL Ltd, 400 MHZ) and a reaction formula are shown below.

¹H-NMR (CDCl₃/TMS, 400 MHZ): δ 1.39 (9H, s), 3.92 (3H, s), 3.94 (3H, s), 6.85 (1H, d, J=8.6 Hz), 7.42 (1H, d, J=20 Hz), 7.55 (1H, dd, J=8.6, 20 Hz).

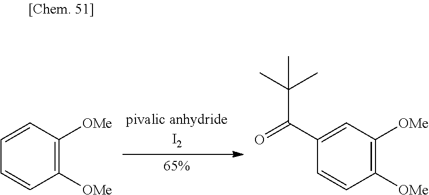 [SMILES: COc1ccccc1OC ; COc1ccc(C(=O)C(C)(C)C)cc1OC]

(Synthesis of 1-(4,5-dimethoxy-2-nitrophenyl)-2,2-dimethyl-1-propanone)
1-(3,4-Dimethoxyphenyl)-2,2-dimethyl-1-propanone (16.3 g, 73.3 mmol) was put into a 200 mL eggplant flask and dissolved in acetic acid (25 mL), fuming nitric acid (45 mL, 1067 mmol) was slowly added dropwise thereto on an ice bath, and the solution was stirred at 0° C. for two hours. A reaction solution was poured into cold water (1500 mL), suction-filtered and sequentially washed with pure water and hexane. The reaction solution was recrystallized (ethanol) by a slow cooling method, and 5.64 g (21.1 mmol, 29%) of a yellow crystal was obtained.

A measurement result of 1H-NMR (JEOL Ltd, 400 MHz) and a reaction formula are shown below.

¹H-NMR (CDCl₃/TMS, 400 MHZ): δ 1.26 (9H, s), 3.98 (3H, s), 3.99 (3H, s), 6.59 (1H, s), 7.70 (1H, s).

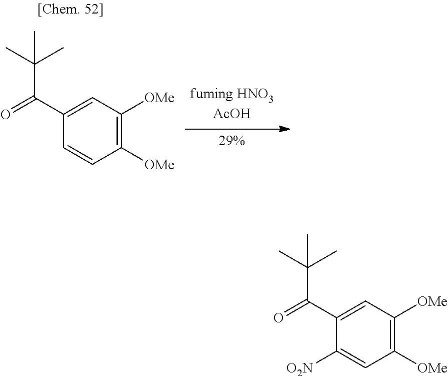 [SMILES: COc1ccc(C(=O)C(C)(C)C)cc1OC ; COc1cc(C(=O)C(C)(C)C)c([N+](=O)[O-])cc1OC]

(Synthesis of 1-(4,5-dimethoxy-2-nitrophenyl)-2,2-dimethylpropanone)

1-(4,5-Dimethoxy-2-nitrophenyl)-2,2-dimethyl-1-propanone (5.64 g, 21.1 mmol) was put into a 300 mL eggplant flask and dissolved in tetrahydrofuran (140 mL) and methanol (84 mL), a small amount of sodium borohydride (2.00 g, 52.8 mmol) was added to each solution on an ice bath, and the solution was then stirred at 0° C. for 30 minutes and further stirred at room temperature for two hours.

After concentration, an organic layer was extracted by adding ethyl acetate (100 mL×4), pure water (150 mL) and 2N hydrochloric acid (35 mL), washed with saturated salt water (150 mL), dried over anhydrous magnesium sulfate, filtered and concentrated, thereby obtaining 5.58 g (20.7 mmol, 98%) of a yellow solid.

A measurement result of 1H-NMR (JEOL Ltd, 400 MHZ) and a reaction formula are shown below.

¹H-NMR (CDCl₃/TMS, 400 MHZ): δ 0.90 (9H, s), 2.05 (1H, d, J=3.6 Hz), 3.94 (3H, s), 3.97 (3H, s), 5.63 (1H, d, J=3.3 Hz), 7.24 (1H, s), 7.45 (1H, s).

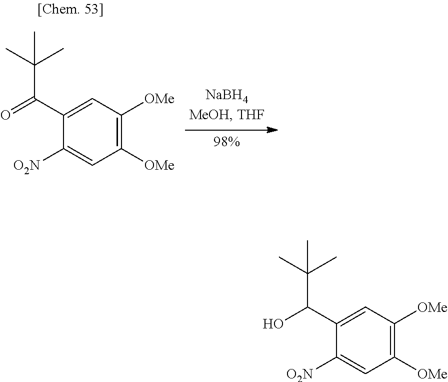 [SMILES: COc1cc(C(=O)C(C)(C)C)c([N+](=O)[O-])cc1OC ; COc1cc(C(O)C(C)(C)C)c([N+](=O)[O-])cc1OC]

(Synthesis of 1-(4,5-dimethoxy-2-nitrophenyl)-2,2-dimethylpropyl (2,5-dioxo-1-pyrrolidinyl)Carbonate)

1-(4,5-Dimethoxy-2-nitrophenyl)-2,2-dimethylpropanone (1.00 g, 3.72 mmol) was put into a 100 mL two-neck eggplant flask and dissolved in dry acetonitrile (15 mL), di(N-succinimidyl) carbonate (1.44 g, 5.63 mmol) and triethylamine (1.56 mL, 11.5 mmol) were added thereto, and the solution was stirred at room temperature for 18 hours under a nitrogen atmosphere.

After concentration, an organic layer was extracted by adding chloroform (80 mL×4), pure water (100 mL) and 2N hydrochloric acid (10 mL), washed with saturated salt water (200 mL), dried over anhydrous magnesium sulfate, filtered and concentrated. The organic layer was purified by silica gel column chromatography (hexane:ethyl acetate=1:1), thereby obtaining 0.65 g (1.59 mmol, 43%) of a yellow powder.

A measurement result of 1H-NMR (JEOL Ltd, 400 MHz) and a reaction formula are shown below.

¹H-NMR (CDCl₃/TMS, 400 MHz): δ 1.02 (9H, s), 2.79 (4H, s), 3.95 (3H, s), 4.04 (3H, s), 6.71 (1H, s), 6.98 (1H, s), 7.60 (1H, s).

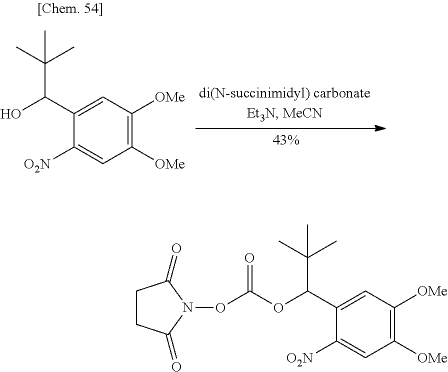 [SMILES: COc1cc(C(O)C(C)(C)C)c([N+](=O)[O-])cc1OC ; COc1cc(C(OC(=O)ON2C(=O)CCC2=O)C(C)(C)C)c([N+](=O)[O-])cc1OC]

(Synthesis of S-octyl O-(1-(4,5-dimethoxy-2-nitrophenyl)-2,2-dimethylpropyl)Carbonate)

1-(4,5-Dimethoxy-2-nitrophenyl)-2,2-dimethylpropyl (2,5-dioxo-1-pyrrolidinyl) carbonate (0.31 g, 0.73 mmol) was put into a 20 mL two-neck eggplant flask and dissolved in dry tetrahydrofuran (10 mL), 4-dimethylaminopyridine (DMAP) (0.37 mg, 2.92 mmol) and 1-octanethiol (0.51 mL, 2.92 mmol) were added thereto, and the solution was stirred at room temperature for 20 hours under a nitrogen atmosphere.

After concentration, an organic layer was extracted by adding chloroform (10 mL×3), pure water (30 mL) and 2N hydrochloric acid (6 mL), washed with saturated salt water (50 mL), dried over anhydrous magnesium sulfate, filtered and concentrated. The organic layer was purified by silica gel column chromatography (hexane:ethyl acetate=8:1), thereby obtaining 0.18 g (0.41 mmol, 56%) of a yellow solid.

A measurement result of 1H-NMR (JEOL Ltd, 400 MHz) and a reaction formula are shown below.

¹H-NMR (CDCl₃/TMS, 400 MHZ): δ 0.87 (3H, t, J=6.8 Hz), 0.97 (9H, s), 1.17-1.39 (10H, m), 1.52-1.63 (2H, m), 2.73-2.88 (2H, m), 3.94 (3H, s), 3.95 (3H, s), 6.77 (1H, s), 6.92 (1H, s), 7.57 (1H, s).

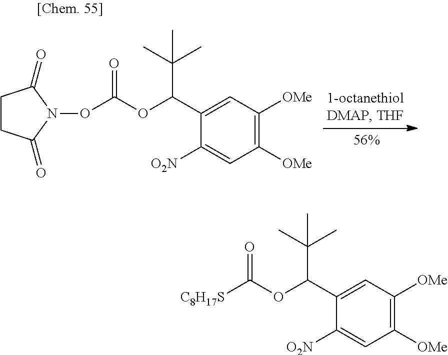

Each of the compounds manufactured in Examples 3 to 9 was dissolved in acetonitrile to prepare a 0.1 mM solution. The solution was irradiated with light having a wavelength of 365 nm at an illuminance of 25 mW/cm² with an ultrahigh pressure mercury lamp through a 365 nm bandpath filter and a water filter for 0, 5, 10, 15, 20, 25 and 30 seconds and measured by HPLC.

The photodegradation rate constant k (s⁻¹) was obtained from the decrease rate of the raw material by assigning the peak area (S₀: area before irradiation with light, Sₜ: area after t seconds from light irradiation) of the raw material obtained by the HPLC measurement. The results are shown in Table 1.

$$ln ( \frac{{S}_{t}}{{S}_{0}} ) = - kt \qquad [ Math . 1 ]$$

TABLE 1

| | | R | k (s⁻¹) |
|---|---|---|---|
| 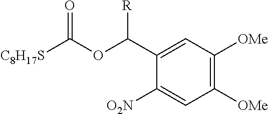 | Example 3 Example 5 Example 7 Example 9 | H Me i-Pr t-Bu | 0.003 0.016 0.017 0.039 |
| 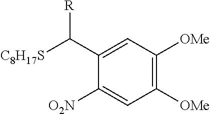 | Example 4 Example 6 Example 8 | H Me i-Pr | 0.019 0.087 0.034 |

It was confirmed that all of the compounds manufactured in Examples 3 to 9 had a photodegradation rate constant of more than 0 and were photodegraded. All of the compounds manufactured in Examples 3 to 9 generated thiol when irradiated with light and were thus capable of selectively patterning thiol at an arbitrary position in the same manner as in Example 2.

In the compounds of Examples 3 to 9, it was possible to confirm that the photodegradation rates improved when the substituent R was introduced into the benzyl site compared with when the compounds were not substituted (R=H). When comparison was made with the same R's, sulfides photodegraded more rapidly than thiocarbonates. It was found that the sulfide having a methyl group (Me) at the benzyl site photodegraded most rapidly.

### Reference Signs List

11 Substrate

10a Photosensitive surface treatment agent

10 Photosensitive surface treatment agent layer

13 Photomask

14 Thiol generation part

12 Thiol non-generation part

15 Catalyst layer

16 Plating layer

17 Insulator layer

18 Plating layer (source electrode)

19 Plating layer (drain electrode)

21 Semiconductor layer

What is claimed is:

1. A photosensitive surface treatment agent comprising:
a compound represented by the following formula (1) or a polymer compound derived from the following formula (1), 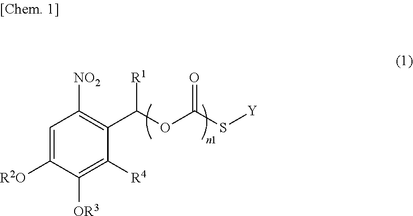
[in the formula (1), R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen atom or a nitro group, n1 is 0 or 1, and Y is one of a polymerizable group-containing group and a linear or branched alkyl group having 1 to 20 carbon atoms].

2. The photosensitive surface treatment agent according to claim 1,
wherein the Y is represented by the following formula (Y1), 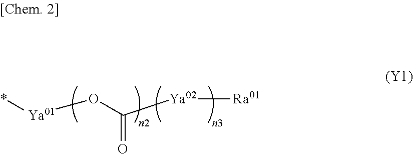
[in the formula (Y1), Ya⁰¹ is an alkylene group having 1 to 10 carbon atoms, Ya⁰² is a group obtained by removing two hydrogen atoms from an aromatic ring having 6 to 15 carbon atoms, Ra⁰¹ is one of a polymerizable group and a linear or branched alkyl group having 1 to 20 carbon atoms, n2 and n3 are each independently 0 or 1, and * means a bonding site to a sulfur element].

3. The photosensitive surface treatment agent according to claim 1,
wherein the Y is represented by the following formula (Y2), 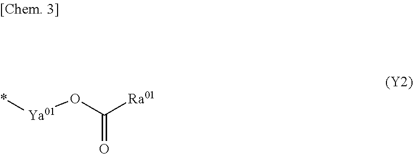
[in the formula (Y2), Ya⁰¹ is an alkylene group having 1 to 10 carbon atoms, Ra⁰¹ is one of a polymerizable group and a linear or branched alkyl group having 1 to 20 carbon atoms, and * means a bonding site to a sulfur element].

4. The photosensitive surface treatment agent according to claim 1,
wherein the Y is represented by the following formula (Y3), 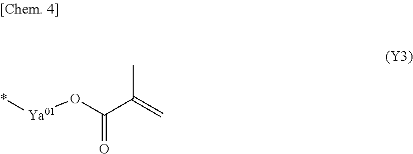
[in the formula (Y3), Ya⁰¹ is an alkylene group having 1 to 10 carbon atoms and * means a bonding site to a sulfur element].

5. The photosensitive surface treatment agent according to claim 1,
wherein the Y is represented by the following formula (Y3-2), 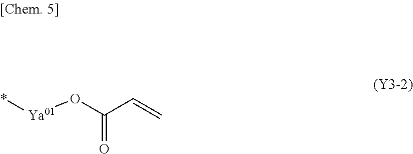
[in the formula (Y3-2), Ya⁰¹ is an alkylene group having 1 to 10 carbon atoms and * means a bonding site to a sulfur element].

6. The photosensitive surface treatment agent according to claim 1,
wherein the Y is represented by the following formula (Y4), 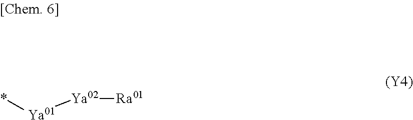
[in the formula (Y4), Ya⁰¹ is an alkylene group having 1 to 10 carbon atoms that may have an ether bond, Ya⁰² is a group obtained by removing two hydrogen atoms from an aromatic ring having 6 to 15 carbon atoms, Ra⁰¹ is one of a polymerizable group and a linear or branched alkyl group having 1 to 20 carbon atoms, and * means a bonding site to a sulfur element].

7. The photosensitive surface treatment agent according to claim 1,
wherein the Y is represented by the following formula (Y5), 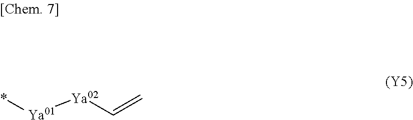
[in the formula (Y5), Ya⁰¹ is an alkylene group having 1 to 10 carbon atoms that may have an ether bond, Ya⁰² is a group obtained by removing two hydrogen atoms from an aromatic ring having 6 to 15 carbon atoms, and * means a bonding site to a sulfur element].

8. The photosensitive surface treatment agent according to claim 1,
wherein the Y is represented by the following formula (Y5-2), 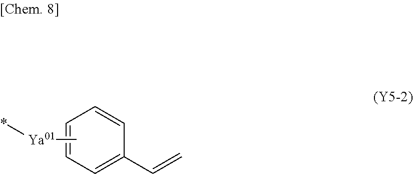
[in the formula (Y5-2), Ya⁰¹ is an alkylene group having 1 to 10 carbon atoms that may have an ether bond and * means a bonding site to a sulfur element].

9. The photosensitive surface treatment agent according to claim 1,
wherein the polymer compound derived from the compound represented by the formula (1) is a polymer compound represented by the following formula (1)-1, 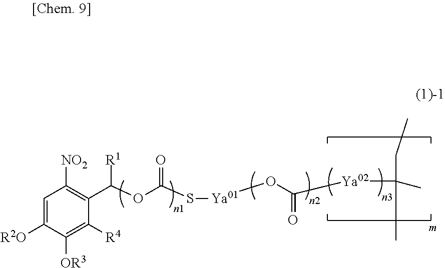
[in the formula (1)-1, R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen or a nitro group, Ya⁰¹ is an alkylene group having 1 to 10 carbon atoms, Ya⁰² is a group obtained by removing two hydrogen atoms from an aromatic ring having 6 to 15 carbon atoms, n1, n2 and n3 are each independently 0 or 1, and m is a natural number].

10. The photosensitive surface treatment agent according to claim 9,
wherein, in the polymer compound derived from the compound represented by the formula (1), a substituent represented by the following formula (1x) bonds to at least one end of a main chain, 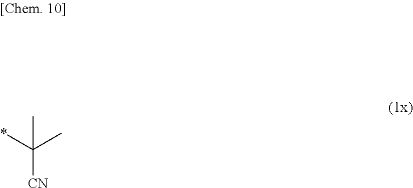 [SMILES: *C(C)(C)C#N]
[* means a bonding site to the end of the main chain of the polymer compound derived from the compound represented by the formula (1)].

11. The photosensitive surface treatment agent according to claim 1,
wherein the polymer compound derived from the formula (1) has a number-average molecular weight of 300 to 100,000.

12. The photosensitive surface treatment agent according to claim 1, further comprising:
cyclopentanone.

13. A laminate comprising:
The photosensitive surface treatment agent according to claim 1.

14. A transistor comprising:
a gate electrode, a source electrode, and a drain electrode; and
the photosensitive surface treatment agent according to claim 1;
wherein the photosensitive surface treatment agent is located under any one of the gate electrode, the source electrode, and the drain electrode.

15. A method for forming a pattern, comprising:
a step of forming a photosensitive resin film over a substrate by applying the photosensitive surface treatment agent according to claim 1;
a step of irradiating the photosensitive resin film with a predetermined pattern light; and
a step of performing electroless plating over at least a part of the photosensitive resin film irradiated with the predetermined pattern light.

16. A method for forming a pattern comprising:
a step of forming a photosensitive resin film over a substrate by applying the photosensitive surface treatment agent according to claim 1;
a step of irradiating the photosensitive resin film with a predetermined pattern light; and
a step of disposing a catalyst for electroless plating in at least a part of the photosensitive resin film irradiated with the predetermined pattern light, and performing electroless plating over the part of the photosensitive resin film.

17. A method for forming a pattern comprising:
a step of forming a photosensitive resin film over a substrate to by applying the photosensitive surface treatment agent according to claim 1;
a step of irradiating the photosensitive resin film with a predetermined pattern light to form a thiol generation region; and
a step of disposing a catalyst for electroless plating in the thiol generation region and performing electroless plating over the part of the photosensitive resin film.

18. A method for manufacturing a transistor comprising:
a step of forming any one or more electrodes of a source electrode, a drain electrode or a gate electrode by the method for forming a pattern according to claims 15.

19. A transistor comprising:
a compound represented by the following formula (1) or a polymer compound derived from the following formula (1), 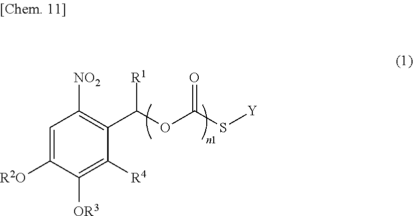
[in the formula (1), R¹ is a hydrogen atom or an alkyl group having 1 to 5 carbon atoms, R² and R³ are each independently an alkyl group having 1 to 3 carbon atoms or a fluoroalkyl group having 1 to 13 carbon atoms, R⁴ is a hydrogen atom or a nitro group, n1 is 0 or 1, and Y is one of a polymerizable group-containing group and a linear or branched alkyl group having 1 to 20 carbon atoms].

20. The transistor according to claim 19,
wherein the compound or the polymer compound has a portion where at least a part of nitrobenzyl groups have been desorbed and a thiol group has been generated.
